(19) United States
(12) Patent Application Publication
(10) Pub. No.: US 2021/0093797 A1
(43) Pub. Date: Apr. 1, 2021

(54) DRUG DELIVERY DEVICE

(71) Applicant: AMGEN INC., Thousand Oaks, CA (US)

(72) Inventors: Emil Finkelstein, Frederiksberg (DK); Soren Forbech Skall, Soborg (DK); Lars Eilertsen, Soborg (DK); Rasmus Øhlenschlæger, Kobenhavn V (DK)

(21) Appl. No.: 17/036,690

(22) Filed: Sep. 29, 2020

Related U.S. Application Data
(60) Provisional application No. 62/908,504, filed on Sep. 30, 2019.

(51) Int. Cl. A61M 5/315; A61M 5/20

(52) U.S. Cl. CPC A61M 5/31581; A61M 2205/581; A61M 5/31535; A61M 5/2033

(57) ABSTRACT

A drug delivery device may include a housing having an opening and a drug storage container including a delivery member with an insertion end configured to extend at least partially through the opening. A biasing member may initially be retained in an energized state, and may be released to drive a plunger to expel a drug from the drug storage container. The plunger may be configured to selectively rotate from an initial rotational position to a second rotational position under a biasing force exerted by the biasing member, and translate linearly in a distal direction to drive the stopper through the drug storage container after rotating from the initial rotational position to the second rotational position. A releaser member may have an initial position wherein the releaser member retains the biasing member in the energized state, and a second position wherein the releaser member generates an audible end-of-dose signal.

### CROSS-REFERENCE TO RELATED APPLICATION

The present application claims the priority of U.S. Provisional Application No. 62/908,504, filed Sep. 30, 2019, entitled “Drug Delivery Device,” which is incorporate by reference herein.

### FIELD OF DISCLOSURE

The present disclosure relates to drug delivery devices, and, more particularly, devices for automatically injecting a drug into a patient.

### BACKGROUND

A general aversion to exposed needles, as well as health and safety issues, have led to the development of drug delivery devices which conceal a needle or other insertion member prior to use and which automate various aspects of an injection process. Such devices offer a variety of benefits as compared with traditional forms of drug delivery including, for example, delivery via a conventional syringe.

A drug delivery device may incorporate various mechanisms to implement various automated features. Such features include automatically covering a needle in a pre-delivery and/or post-delivery state, providing an interface for a user to activate a drive mechanism, indicating to the user that drug delivery is complete, among other features. Typically a drug delivery device will incorporate a separate or independently operable mechanism to realize each of its automated features. As a consequence, with each added feature, the mechanical complexity of the device tends to increase. This, in turn, can increase the size of the device, which can make it cumbersome for the user to handle, as well as increase manufacturing costs and timeframes. As the demand grows for drug delivery devices with greater ease of use and safety, finding a way to incorporate more automated features without adding undue complexity to the drug delivery device presents various design and manufacturing challenges.

The present disclosure sets forth drug delivery devices embodying advantageous alternatives to existing drug delivery devices, and that may address one or more of the challenges or needs mentioned herein.

### SUMMARY

One aspect of the present disclosure provides a drug delivery device including a housing, a drug delivery container fixed relative to the housing, a biasing member, and a plunger operably coupled to the plunger biasing member. The drug storage container may include an interior surface and a stopper slidable along the interior surface. The plunger may be configured to: (i) selectively rotate from an initial rotational position to a second rotational position under a biasing force exerted by the biasing member, and (ii) translate linearly in a distal direction to drive the stopper through the drug storage container after rotating from the initial rotational position to the second rotational position.

Another aspect of the present disclosure provides a drug delivery device including a housing having an opening, a drug storage container, a guard moveably positioned adjacent to the opening, a plunger, a plunger biasing member, and a releaser member. The drug storage container may include a delivery member having an insertion end configured to extend at least partially through the opening. The plunger may be moveable in a distal direction to expel a drug from the drug storage container through the delivery member. The releaser member may be operably coupled to the guard and the plunger. Furthermore, the releaser member may be configured to rotate from an initial rotational position to a second rotational position under a biasing force exerted by the plunger biasing member.

An additional aspect of the present disclosure provides a drug delivery device including a housing, a drug storage container, a plunger, a plunger biasing member initially retained in an energized state, and an indicator. The drug storage container may include a delivery member having an insertion end configured to extend at least partially through the opening. Releasing the plunger biasing member may drive the plunger in a distal direction to expel a drug from the drug storage container through the delivery member. The indicator may have an initial position wherein the indicator retains the plunger biasing member in the energized state, and a second position wherein the indicator generates an audible signal indicating an end of drug delivery.

Another aspect of the present disclosure provides a housing having an opening, a drug storage container, a plunger, and a plunger biasing member. The drug storage container may include a delivery member having an insertion end configured to extend at least partially through the opening. The plunger may have an inner surface defining an axial chamber. The plunger biasing member may be disposed at least partially within the axial chamber of the plunger and may be initially retained in an energized state. Releasing the plunger biasing member may drive the plunger in a distal direction to expel a drug from the drug storage container through the delivery member.

An additional aspect of the present disclosure provides a housing having an opening, a drug storage container, a guard moveable positioned adjacent to the opening, a plunger, a plunger biasing member, and a releaser member. The drug storage container may include a delivery member having an insertion end configured to extend at least partially through the opening. The drug storage container may be coupled with the housing such as to resist relative movement therebetween. The plunger may be moveable in a distal direction to expel a drug from the drug storage container through the delivery member. The releaser member may be operably coupled to the guard and the plunger. Further, the releaser member may be configured to utilize inertial forces from a user to drive the housing and the drug storage container toward an injection site of the user.

A further aspect of the present disclosure provides a drug delivery device including a housing having an opening, a drug storage container, a plunger, a plunger biasing member, and a brake member. The drug storage container may include a body portion defining a longitudinal axis and a delivery member having an insertion end configured to extend at least partially through the opening during a delivery state. The plunger may be moveable in a distal direction to expel a drug from the drug storage container through the delivery member. The plunger biasing member may be configured to urge the plunger in the distal direction. The brake member may be operably coupled to the plunger. Movement of the plunger in the distal direction may cause the plunger and/or the brake member to rotate about the longitudinal axis.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 is a perspective view of a drug delivery device according to an embodiment of the present disclosure.

FIG. 2 is cross-sectional view of the drug delivery device in FIG. 1.

FIG. 3 is an exploded assembly view of the drug delivery device in FIG. 2.

FIGS. 4 and 5 are different perspective views of a plunger guide illustrated in FIG. 2.

FIGS. 6 and 7 are different perspective views of a releaser member depicted in FIG. 2.

FIG. 8 is a partial perspective view of a plunger, a plunger biasing member, and a plunger guide shown in FIG. 2.

FIG. 9A is a cross-sectional view taken along line Z-Z in FIG. 9B.

FIG. 9B is perspective view of a plunger retaining arrangement prior retraction of a guard member. In FIG. 9B, the releaser member is illustrated as being semi-transparent. Also, in FIG. 9B, the guard extension and the guard biasing member are omitted for clarity.

FIG. 9C is a perspective view of a distal end of the plunger retaining arrangement in FIG. 9B. In FIG. 9C, each of the guard and the guard extension is illustrated as being semi-transparent. Also, in FIG. 9C, the guard biasing member, the plunger, and the plunger guide are omitted for clarity.

FIG. 9D is a cross-sectional view taken along line Y-Y in FIG. 9C.

FIG. 9E is perspective view of a proximal end of the retaining arrangement in FIG. 9B. In FIG. 9E, the releaser member is illustrated as being semi-transparent. Also, in FIG. 9E, the guard biasing member is omitted for clarity.

FIG. 10A is a cross-sectional view taken along line X-X in FIG. 10B.

FIG. 10B is perspective view of the plunger retaining arrangement in the moments after the guard member has moved to the retracted position. In FIG. 10B, the releaser member is illustrated as being semi-transparent. Also, in FIG. 10B, the guard extension and the guard biasing member are omitted for clarity.

FIG. 10C is a perspective view of a distal end of the plunger retaining arrangement in FIG. 10B. In FIG. 10C, each of the guard and the guard extension is illustrated as being semi-transparent. Also, in FIG. 10C, the guard biasing member, the plunger, and the plunger guide are omitted for clarity.

FIG. 10D is a cross-sectional view taken along line W-W in FIG. 10C.

FIG. 11A is a cross-sectional view taken along line V-V in FIG. 11B.

FIG. 11B is perspective view of the plunger retaining arrangement at the start of drug delivery. In FIG. 11B, the releaser member is illustrated as being semi-transparent. Also, in FIG. 11B, the guard extension and the guard biasing member are omitted for clarity.

FIG. 11C is a perspective view of a distal end of the plunger retaining arrangement in FIG. 11B. In FIG. 11C, each of the guard and the guard extension is illustrated as being semi-transparent. Also, in FIG. 11C, the guard biasing member, the plunger, and the plunger guide are omitted for clarity.

FIG. 11D is a cross-sectional view taken along line U-U in FIG. 11C.

FIG. 11E is perspective view of a proximal end of the retaining arrangement in FIG. 11B. In FIG. 11E, the releaser member is illustrated as being semi-transparent. Also, in FIG. 11E, the guard biasing member is omitted for clarity.

FIG. 12A is a cross-sectional view taken along line T-T in FIG. 12B.

FIG. 12B is perspective view of the plunger retaining arrangement at the end of drug delivery. In FIG. 12B, the releaser member is illustrated as being semi-transparent. Also, in FIG. 12B, the guard extension and the guard biasing member are omitted for clarity.

FIG. 12C is a perspective view of a distal end of the plunger retaining arrangement in FIG. 12B. In FIG. 12C, each of the guard and the guard extension is illustrated as being semi-transparent. Also, in FIG. 12C, the guard biasing member, the plunger, and the plunger guide are omitted for clarity.

FIG. 12D is a cross-sectional view taken along line S-S in FIG. 12C.

FIG. 12E is perspective view of a proximal end of the retaining arrangement in FIG. 12B. In FIG. 12E, the releaser member is illustrated as being semi-transparent. Also, in FIG. 12E, the guard biasing member is omitted for clarity.

FIG. 13 is a perspective view of a drug delivery device according to another embodiment of the present disclosure.

FIG. 14 is a perspective view of the drug delivery device in FIG. 13, with a removable cap removed.

FIGS. 15 and 16 are different side views of the drug delivery device in FIG. 13.

FIG. 17A is a cross-sectional view of a drug delivery device according to another embodiment of the present disclosure.

FIG. 17B is an enlarged view of a proximal end of the drug delivery device illustrated in FIG. 17A.

FIG. 18A is a cross-sectional view of a drug delivery device according to another embodiment of the present disclosure.

FIG. 18B is an enlarged view of a proximal end of the drug delivery device illustrated in FIG. 18A.

FIG. 19A is a cross-sectional view of a drug delivery device according to another embodiment of the present disclosure.

FIG. 19B is an enlarged view of a proximal end of the drug delivery device illustrated in FIG. 19A.

FIG. 20 is a cross-sectional view of a drug delivery device according to another embodiment of the present disclosure.

FIG. 21 is a cross-sectional view of a drug delivery device according to another embodiment of the present disclosure.

### DETAILED DESCRIPTION

The present disclosure generally relates to drug delivery devices operable by a user for administering a drug, or in the case where a patient is the user, self-administering a drug. Various features are disclosed to facilitate safe and proper handling of the drug delivery device, including handling the drug delivery device after it has been used to deliver its payload. Such features include, but are not limited to, an indicator for signaling to the user that drug delivery is complete and a drive mechanism activatable by pressing the drug delivery device against the patient's skin at the injection site. These features and others work together and/or interact with one another in synergistic ways so as to limit the number of moving parts and/or complexity of the drug delivery device. Furthermore, certain features described herein exploit a biasing force exerted by a plunger biasing member and/or a guard biasing member for actuation purposes, thereby reducing any force that must be applied by the user and/or alleviating a need to incorporate a dedicated energy source for implementing said feature. These and other advantages will be apparent to one of ordinary skill in the art reviewing the present disclosure.

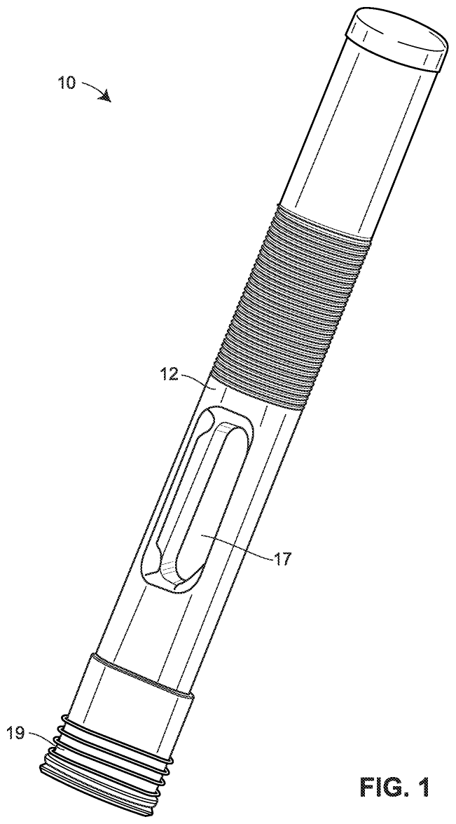

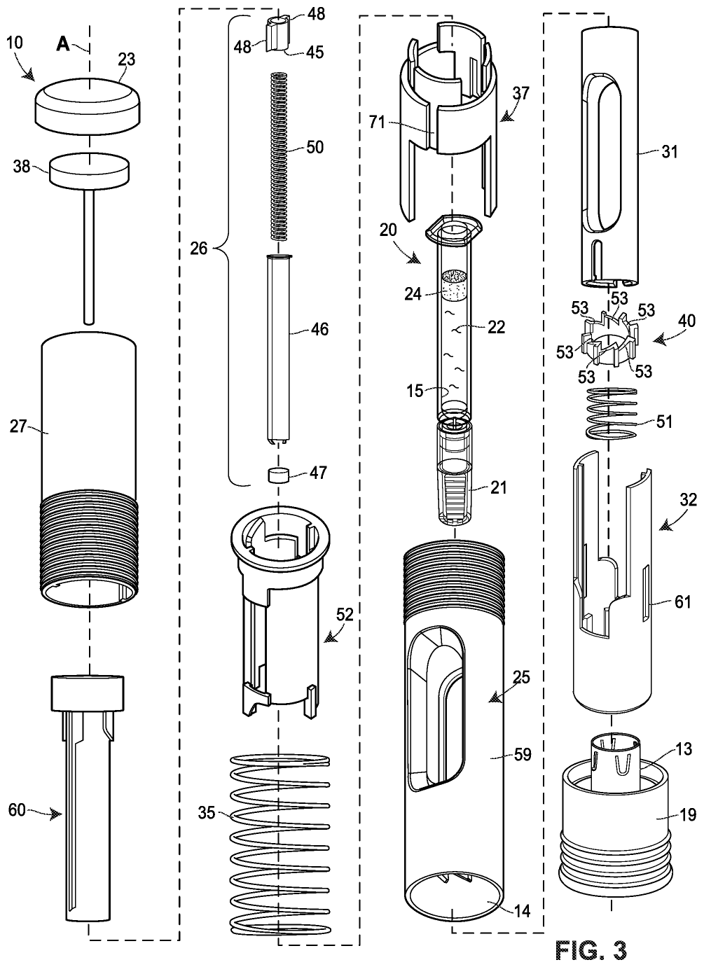

FIGS. 1-3 illustrate several views of an embodiment of a drug delivery device 10 for delivering a drug, which may also be referred to herein as a medicament or drug product. The drug may be, but is not limited to, various biologicals such as peptides, peptibodies, or antibodies. The drug may be in a fluid or liquid form, although the disclosure is not limited to a particular state.

Various implementations and configurations of the drug delivery device 10 are possible. The present embodiment of the drug delivery device 10 is configured as a single-use, disposable injector. In other embodiments, the drug delivery device 10 may be configured as multiple-use reusable injector. The drug delivery device 10 is operable for self-administration by a patient or for administration by caregiver or a formally trained healthcare provider (e.g., a doctor or nurse). The present embodiment of the drug delivery device 10 takes the form of an autoinjector or pen-type injector, and, as such, may be held in the hand of the user over the duration of drug delivery.

The configuration of various components included in the drug delivery device 10 may depend on the operational state of the drug delivery device 10. The drug delivery device 10 may have a pre-delivery or storage state, a delivery or dosing state, and a post-delivery state, although fewer or more states are also possible. The pre-delivery state may correspond to the configuration of the drug delivery device 10 subsequent to assembly and prior to activation by the user. In some embodiments, the pre-delivery state may exist in the time between when the drug delivery device 10 leaves a manufacturing facility and when a patient or user activates a drive mechanism 30 of the drug delivery device 10. This includes the moments in time after the user has removed the drug delivery device 10 from any secondary packaging and prior to positioning the drug delivery device 10 against the injection site. The delivery state may correspond to the configuration of the drug delivery device 10 while drug delivery, also referred to herein as dosing, is in progress. The post-delivery state may correspond to the configuration of the drug delivery device 10 after drug delivery is complete and/or when a stopper is arranged in an end-of-dose position in a drug storage container.

The drug delivery device 10 includes an outer casing or housing 12. In some embodiments, the housing 12 may be sized and dimensioned to enable a person to grasp the injector 10 in a single hand. The housing 12 may have a generally elongate shape, such as a cylindrical shape, and extend along a longitudinal axis A between a proximal end and a distal end. An opening 14 may be formed in the distal end to permit an insertion end 28 of a delivery member 16 to extend outside of the housing 12. A transparent or semi-transparent inspection window 17 may be positioned in a wall of the housing 12 to permit a user to view component(s) inside the drug delivery device 10, including a drug storage container 20. Viewing the drug storage container 20 through the window 17 may allow a user to confirm that drug delivery is in progress and/or complete. A removable cap 19 may cover the opening 14 prior to use of the drug delivery device 10, and, in some embodiments, may including a gripper 13 configured to assist with removing a sterile barrier 21 (e.g., a rigid needle shield (RNS), a flexible needle shield (FNS), etc.) mounted on the insertion end 28 of the delivery member 16. The gripper 13 may include one or more inwardly protruding barbs or arms that frictionally or otherwise mechanically engage the sterile barrier 21 to pull the sterile barrier 21 with the removable cap 19 when the user separates the removable cap 19 from the housing 12. Thus, removing the removable cap 19 has the effect of removing the sterile barrier 21 from the delivery member 16.

In the present embodiment, the housing 12 is defined by three separate and interconnected structures: a rear end cap 23 at the proximal end of the drug delivery device 10; a front housing 25 at the distal end of the drug delivery device 10 and which includes the opening 14; and a rear housing 27 positioned between and rigidly connecting the rear end cap 23 and the front housing 25. The front housing 25 and the rear housing 27 each may have a hollow and generally cylindrical or tubular shape, and the rear end cap 23 may have a generally hemispherical shape or a hollow cylindrical shape with an open end and a closed off end. In some embodiments, the rear end cap 23 and the rear housing 27, and any components to be positioned therein, may be assembled together to define a rear sub-assembly. Meanwhile the front housing 25 and any components to be positioned therein may be assembled together to define a front sub-assembly. In some embodiments, the rear and front sub-assemblies are assembled independently of each other and then later combined with one another, as well as with the drug storage container 20, to form the fully-assembled drug delivery device 10. In certain such embodiments, some or all of the foregoing phases of assembly may occur in different manufacturing facilities or environments. In alternative embodiments, the housing 12 may be constructed in one piece, such that the housing 12 is defined by a single, monolithic structure.

The drug storage container 20 is disposed within an interior space of the housing 12 and is configured to contain a drug 22. The drug storage container 20 may be pre-filled and shipped, e.g., by a manufacturer, to a location where the drug storage container 20 is combined with a remainder of the drug delivery device 10. The housing 12 may be pre-loaded with the drug storage container 20, e.g., by a manufacturer, or alternatively, loaded with the drug storage container 20 by a user prior to use of the drug delivery device 10. The drug storage container 20 may include a rigid wall defining an internal bore or reservoir. The wall may be made of glass or plastic. A stopper 24 may be moveably disposed in the drug storage container 20 such that it can move in a distal direction along the longitudinal axis A between proximal end and a distal end of the drug storage container 20. The stopper 24 may be constructed of rubber or any other suitable material. The stopper 24 may slidably and sealingly contact an interior surface 15 of the wall of the drug storage container 20 such that the drug 22 is prevented or inhibited from leaking past the stopper 24 when the stopper 24 is in motion. Distal movement of the stopper 24 expels the drug 22 from the reservoir of the drug storage container 20 into the delivery member 16. The proximal end of the drug storage container 20 may be open to allow a plunger 26 to extend into the drug storage container 20 and push the stopper 24 in the distal direction. In the present embodiment, the plunger 26 and the stopper 24 are initially spaced from each other by a gap. Upon activation of a drive mechanism 30, the plunger 26 moves in the distal direction to close the gap and comes into contact with the stopper 24. Subsequent distal movement of the plunger 26 drives the stopper 24 in the distal direction to expel the drug 22 from the drug storage container 20. In alternative embodiments, the stopper 24 and the plunger 26 may initially be in contact with one another or coupled to one another, e.g., via a threaded coupling, such that they move together jointly from the start of movement of the plunger 26. Once the stopper 24 is in motion, it may continue to move in the distal direction until it contacts a proximally-facing portion of the interior surface 15 of the wall of the drug storage container 20. This position of the stopper 24 may be referred to as the end-of-dose or end-of-delivery position, and may correspond to when delivery of the drug 22 to the patient is complete or substantially complete.

In some embodiments, a volume of the drug 22 included in the reservoir of the drug storage container 20 may be equal to 1 mL, or equal to approximately (e.g., ±10%) 1 mL, or equal to 2.5 mL, or equal to approximately (e.g., ±10%) 2.5 mL, or less than or equal to approximately (e.g., ±10%) 2 mL, or less than or equal to approximately (e.g., ±10%) 3 mL, or less than or equal to approximately (e.g., ±10%) 4 mL, or less than approximately (e.g., ±10%) 5 mL, or less than or equal to approximately (e.g., ±10%) 10 mL, or within a range between approximately (e.g., ±10%) 1-10 mL, or within a range between approximately (e.g., ±10%) 1-5 mL, or within a range between approximately (e.g., ±10%) 1-4 mL, or within a range between approximately (e.g., ±10%) 1-3 mL, or within a range between approximately (e.g., ±10%) 1-2.5 mL.

The delivery member 16 is connected or operable to be connected in fluid communication with the reservoir of the drug storage container 20. A distal end of the delivery member 16 may define the insertion end 28 of the delivery member 16. The insertion end 28 may include a sharpened tip of other pointed geometry allowing the insertion end 28 to pierce the patient's skin 5 and subcutaneous tissue during insertion of the delivery member 16. The delivery member 16 may be hollow and have an interior passageway. One or more openings may be formed in the insertion end 28 to allow drug to flow out of the delivery member 16 into the patient.

In the present embodiment, the drug storage container 20 is a pre-filled syringe and has a staked, hollow metal needle for the delivery member 16. Here, the needle is fixed relative to the wall of the drug storage container 20 and is in permanent fluid communication with the reservoir of the drug storage container 20. In other embodiments, the drug storage container 20 may be a needle-less cartridge, and, as such, initially may not be in fluid communication with the delivery member 16. In such embodiments, the drug storage container 20 may move toward a proximal end of the delivery member 16, or vice versa, during operation of the drug delivery device 10 such that the proximal end of the delivery member 16 penetrates through a septum covering an opening in the drug storage container 20 thereby establishing fluid communication between the reservoir of the drug storage container 20 and the delivery member 16.

The drug storage container 20 may be fixed relative to the housing 12 such that the drug storage container 20 does not move relative to the housing 12 once installed in the housing 12. As such, the insertion end 28 of the delivery member 16 extends permanently through the opening 14 in the housing 12 in the pre-delivery, delivery, and post-delivery states. In the present embodiment, a container holder 31 fixes the position of the drug storage container 20 within the housing 12. The container holder 31 may have a hollow and generally cylindrical or tubular shape, and the drug storage container 20 may be disposed partially or entirely within the container holder 31. A distal end of the container holder 31 may include an inwardly protruding flange 33 abutting against a neck of the drug storage container 20, thereby preventing distal movement of the drug storage container 20. The container holder 31 may be fixedly attached to the housing 12 such that the container holder 31 is prevented from moving relative to the housing 12 during operation of the drug delivery device 10.

In alternative embodiments, the drug storage container 20 may be moveably coupled to the housing 12 such that the drug storage container 20 is able to move relative to the housing 12 during operation of the drug delivery device 10. In certain such alternative embodiments, the insertion end 28 of the delivery member 16 may be retracted within the opening 14 in the housing 12 in the pre-delivery state. Subsequently, during operation of the injection device 10, the insertion end 28 of the delivery member 16 may be deployed through the opening 14 in the housing 12 for insertion into the patient. This motion may, in some embodiments, be the result of the drug storage container 20 having been driven in the distal direction relative to the housing 12.

The plunger 26 may have a hollow and generally cylindrical or tubular shape. The plunger 26 may include an annular wall 39 with an outer surface 41 and an inner surface 43. The inner surface 43 may define an interior space sized to receive a plunger biasing member 50 therein. It is generally desirable for a thickness of the annular wall 39 to be minimized, to the extent possible without compromising the integrity of the plunger 26, so as to maximize an inner diameter of the plunger 26. This allows a larger diameter plunger biasing member 50 to fit within the interior space of the plunger 26, which, in turn, allows for a more powerful plunger biasing member 50. As described below in more detail, the plunger 26 may be configured to selectively rotate relative to the housing 12 and translate linearly relative to the housing 12 during operation of the drug delivery device 10.

The plunger 26 may be constructed of multiple, interconnected pieces, or alternatively, have a one-piece construction. In the present embodiment, the plunger 26 is constructed of three separate and interconnected structures: a top ring 45 defining a proximal end of the plunger 26; a base 47 defining a distal end of the plunger 26; and a hollow rod 46 positioned between and rigidly connecting the top ring 45 and the base 47. The positions of the top ring 45, the hollow rod 46, and the base 47 may be fixed relative to each other such that these components are immoveable relative to each other. The top ring 45, the hollow rod 46, and the base 47 may each have an annular construction and be centered about the longitudinal axis A. The top ring 45 and the hollow rod 46 may each have a respective central opening extending from end to end of the component to define an axial chamber; whereas, the base 47 may have a central opening extending through the proximal end of the base 47 but which is closed off at the distal end of the base 47. The closed off end of the base 47 may define seat or abutment surface for the plunger biasing member 50. In alternative embodiments, the central opening may extend through the base 47 from end to end. In such alternative embodiments, an inner diameter of the central opening of the base 47 may be smaller than an outer diameter of the plunger biasing member 50 such that the base 47 retains a distal end of the plunger biasing member 50 within the plunger 26. When the drive mechanism 30 is activated, the base 47 may be the portion of the plunger 46 that comes into contact with the stopper 24 to push the stopper 24 in the distal direction.

The top ring 45 may include one or more flanges or projections 48 which extend radially outwardly from a central portion of the top ring 45. Each of the projections 48 may include a distally facing camming surface 49. As described below in more detail, the distally facing camming surface 49 may interact with a counterpart camming surface on a plunger guide 60 in order to release the plunger biasing member 50. In some embodiments, the distally facing camming surface 49 may arranged at angle relative to, or is otherwise non-parallel to, an imaginary plane perpendicular to the longitudinal axis A.

In some embodiments, the top ring 45 and/or the base 47 may be constructed of a different material than the hollow rod 46. In some embodiments, the top ring 45 and/or the base 47 made be constructed of plastic whereas the hollow rod 46 may be constructed of metal. So configured, the plastic material used for the top ring 45 may facilitate the camming action described below by providing sliding friction, the plastic material used for the base 47 may help absorb or attenuate any shock or vibrations associated with base 47 striking the stopper 24. The metal material used for the hollow rod 46 may provide sufficient rigidity to avoid buckling under the biasing force exerted by the plunger biasing member 50. In alternative embodiments, the top ring 45, hollow rod 46, and/or base 47 may be made of the same material, including, for example, metal or plastic. In certain such embodiments, the top ring 45, hollow rod 46, and base 47 may be integrally formed in one piece so as to define single, monolithic structure.

The drug delivery device 10 may further include a guard mechanism for preventing contact with the insertion end 28 of the delivery member 16 when the drug delivery device 10 is not being used to administer an injection. The guard mechanism may include a guard member 32 moveably disposed at the distal end of the housing 12 adjacent to the opening 14. The guard member 32 may have a hollow and generally cylindrical or tubular shape centered about the longitudinal axis A, and may have a proximal end received within the housing 12. The guard member 32 may be configured to move relative to the housing 12 between an extended position wherein a distal end of the guard member 32 extends through the opening 14 in the housing 12 and a retracted position wherein the distal end of the guard member 32 is retracted, fully or partially, into the opening 14 in the housing 12. Additionally or alternatively, the guard member 32 may be configured to move from the retracted position to the extended position. When moving from the extended position to the retracted position, the guard member 32 may translate linearly in the proximal direction; and when moving from the retracted position to the extended position, the guard member 32 may translate linearly in the distal direction. In at least the extended position, the guard member 32 may extend beyond and surround the insertion end 28 of the delivery member 16. In embodiments where the delivery member 16 protrudes from the opening 14 in the housing 12 in the pre-delivery or storage state, moving the guard member 32 from the extended position to the retracted position, e.g., by pressing the distal end of the guard member 32 against the patient's skin at the injection site, may result in the insertion end 28 of the delivery member 16 being inserted into the patient's skin.

For example, the delivery device 10 may utilize inertial design, rather than a spring driven design, to insert the needle into the patient's subcutaneous tissue. As a more specific example, when the patient presses the distal end of the guard member 32 against the patient's skin at the injection site, the delivery device 10 housing 12 may advance toward the injection site. As the patient presses down a predetermined distance or with a predetermined force, the delivery device 10 achieves a quick release to harness the energy stored in the patient's muscles while compressing the needle cover and its spring to a defined release point. The release mechanism is designed such that the resulting needle insertion speed exceeds the patient's reaction speed, and the combination of this speed and the device's mass cause the needle to quickly and fully penetrate the skin to the subcutaneous depth. Compared to known injectors, where the entire primary container is moved forward with respect to the housing, this embodiment prevents relative movement between the drug storage container 20 and the housing and therefore provides a simplified, more robust design.

In some embodiments, the guard member 32 may be rotationally fixed relative to the housing 12. Therefore, although the guard member 32 may able to translate linearly relative to the housing 12, the guard member 32 may be prevented from rotating relative to the housing 12. To achieve this effect, in some embodiments, one or more longitudinal slots 61 may be formed in a wall of the guard member 32 and may be parallel to the longitudinal axis A. Each longitudinal slot 61 may be dimensioned to matingly or snugly receive a projection or pin 63 extending radially inwardly from the front housing 25. Each pin 63 may slidably engage a surface defining a respective one of the longitudinal slots 61 when the guard member 32 translates linearly along the longitudinal axis A relative to the front housing 25. The pin 63, however, abuts against that same surface to prevent rotation of the guard member 32 relative to the front housing 25 if any rotational forces are exerted on the guard member 32. In alternative embodiments, the pin-and-slot arrangement may be reversed, such that the guard member 32 has one or more radially outwardly extending pins and the front housing 25 has one or more slots or other recesses to matingly or snugly receive the one or more pins.

The guard mechanism may further include a guard biasing member 35 and a guard extension 37. The guard extension 37 may be positioned proximal to the guard member 32; and the guard biasing member 35 may be positioned proximal to the guard extension 37. The guard extension 37 may have a hollow and generally cylindrical or tubular shape centered about the longitudinal axis A. Furthermore, the guard extension 37 may be moveable in a linear direction along the longitudinal axis A relative to the housing 12. In the present embodiment, the guard extension 37 is a separate structure from the guard member 32. However, in alternative embodiments, the guard extension 37 and the guard member 32 may be integrally formed in one piece to define a single, monolithic structure. In such alternative embodiments, the proximal end of the guard member 32 may correspond to the guard extension 37.

Similar to the guard member 32, the guard extension 37 may be rotationally fixed relative to the housing 12. Therefore, although the guard extension 37 may able to translate linearly relative to the housing 12, the guard extension 37 may be prevented from rotating relative to the housing 12. To achieve this effect, in some embodiments one or more longitudinal slots 71 may be formed in a wall of the guard extension 37 and may be parallel to the longitudinal axis A. Each longitudinal slot 71 may be dimensioned to matingly or snugly receive a projection or pin (not illustrated) extending radially inwardly from the housing 12, such as, e.g., the rear housing 23 and/or the front housing 25. Each pin may slidably engage a surface defining a respective longitudinal slot 71 when the guard extension 37 translates linearly along the longitudinal axis A relative to the housing 12. The pin, however, abuts against that same surface to prevent rotation of the guard extension 37 relative to the housing 12 if any rotational forces are exerted on the guard extension 37. In alternative embodiments, the pin-and-slot arrangement may be reversed, such that the guard extension 37 has one or more radially outwardly extending pins and the housing 12 has one or more slots or other recesses to matingly or snugly receive the one or more pins.

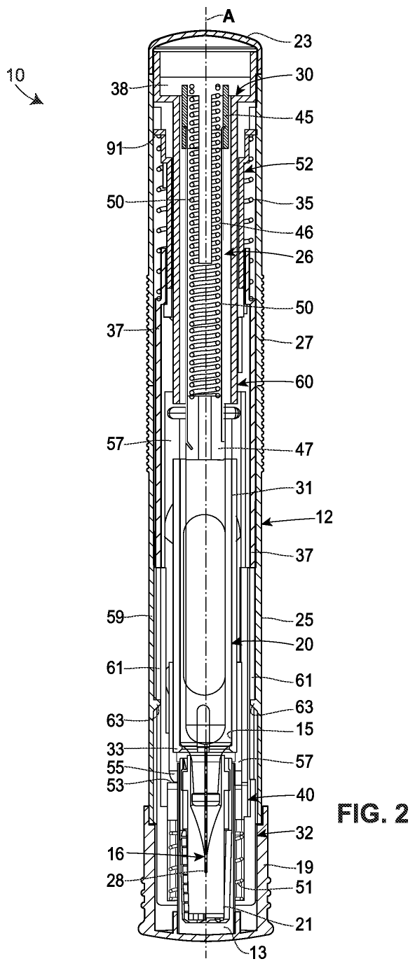

The guard biasing member 35 may be positioned between and in contact with the guard extension 37 and a releaser member 52. The guard biasing member 35 may be configured to bias or urge the guard extension 37 in the distal direction and bias or urge the releaser member 52 in the proximal direction. The guard biasing member 35 may initially be in an energized (e.g., compressed) state such that it exerts a biasing force on the guard extension 37 and a biasing force on the releaser member 52 in the pre-delivery state. In some embodiments, a distal end of the guard extension 37 is initially in contact with a proximal end of the guard member 32, as seen in FIG. 2. As a consequence, the guard extension 37 transfers a biasing force of the guard biasing member 35 to the guard member 32, such that the guard biasing member 35 biases or urges the guard member 32 toward the extended position. A user may overcome the biasing force by pressing the guard member 32 against the injection site. In doing so, the guard member 32 and the guard extension 37 move jointly in the proximal direction until, for example, the guard member 32 reaches the retracted position. When the injection is complete and the drug delivery device 10 is lifted off of the injection site, the guard biasing member 35 may push the guard extension 37 so that the guard extension 37 and the guard member 32 move jointly in the distal direction. This motion returns the guard member 32 to the extended position, which has the effect of covering the insertion end 28 of the deliver member 16. In some embodiments, the guard biasing member 35 may include a compression spring (e.g., a helical compression spring). Furthermore, in embodiments where the plunger biasing member 50 also includes a compression spring, the guard biasing member 35 may disposed around and/or have a larger diameter than the plunger biasing member 50.

In alternative embodiments, the distal end of the guard extension 37 may initially be spaced in the proximal direction from the proximal end of the guard member 32 by a gap. As a consequence, the guard biasing member 35 may not bias the guard member 32 toward the extended position in the pre-delivery state. When the guard member 32 retracts in the proximal direction and comes into contact with the guard extension 37, only then may the guard biasing member 35 exert the biasing force on the guard member 32 urging it toward the extended position. In such alternative embodiments, a lock ring biasing member 51, described below, may solely be relied upon to bias the guard member 32 toward the extended position in the pre-delivery state.

After drug delivery is complete and the guard member 32 has been re-deployed to the extended position, it may be desirable to lock the guard member 32 in the extended position to prevent subsequent user contact with the insertion end 28 of the delivery member 16 and/or to prevent re-use of the drug delivery device 10. Pursuant to these ends, some embodiments of the drug delivery device 10 may include a lock ring 40 configured to selectively rotate, depending on the axial position of the guard member 32, in order to lock the guard member 32 in the extended position once the guard member 32 has moved from the retracted position to the extended position. In the present embodiment, the lock ring 40 is centered and rotates about the longitudinal axis A. As illustrated in FIG. 2, a proximal end of the lock ring 40 may be in contact with the container holder 31 and the distal end of the lock ring 40 may be disposed at least partially within the guard member 32. The lock ring biasing member 51 may be positioned in the axial direction between a distally facing surface of the lock ring 40 and a proximally facing surface of the guard member 32. The lock ring biasing member 51 may initially be in a compressed or energized state such that it biases the lock ring 40 and the guard member 32 away from each other. As such, the lock ring biasing member 51 may exert a biasing force urging the guard member 32 toward the extended position, as well as exert a biasing force urging the proximal end of the lock ring 40 against the container holder 31. In some embodiments, the lock ring biasing member 51 may include a compression spring (e.g., a helical compression spring).

Rotation of the lock ring 40 may be achieved by a camming arrangement between the lock ring 40 and the container holder 31. In some embodiments, the proximal end of the lock ring 40 may include one or more camming surfaces 53 configured to slidably engage one or more corresponding camming surfaces 55 included on an inner annular wall 57 of the front housing 25. The inner annular wall 57 of the front housing 25 may be centered about the longitudinal axis A and may be cantilevered radially inwardly from an outer annular wall 59 of the front housing 25 such that an annular gap exists between the inner annular wall 57 and the outer annular wall 59 of the front housing 25. This configuration may allow for the guard member 32 to slide into the annular gap between the inner and outer walls 57 and 59 during retraction. In some embodiments, the camming surfaces 53 of the lock ring 40 may have a generally saw tooth appearance when viewed in the radial direction from the longitudinal axis A. Furthermore, the camming surfaces 53 may be disposed around the longitudinal axis A such that each camming surface 53 is located at different angular position around the longitudinal axis A. Similarly, the camming surfaces 55 on the container holder 31 may have a generally saw tooth appearance when viewed in the radial direction from the longitudinal axis A. Furthermore, the camming surfaces 55 may be disposed around the longitudinal axis A such that each camming surface 55 is located at different angular position around the longitudinal axis A.

When pressed against one another, the camming surfaces 53 and 55 may convert linear motion into a combination of rotational motion and linear motion. More particularly, when the lock ring 40 moves in the proximal direction along the longitudinal axis A, each of the camming surfaces 53 may slide against a respective one of the camming surfaces 55. This interaction may convert the proximal linear movement of the lock ring 40 into a combination of rotational movement of the lock ring 40 about the longitudinal axis A and proximal linear movement of the lock ring 40 along the longitudinal axis A. Throughout movement of the lock ring 40, the inner annular wall 57 of the front housing 25 remains stationary relative to a remainder of the front housing 25. So configured, the inner annular wall 57 of the front housing 25 functions as a cam and the lock ring 40 as a cam follower.

The biasing force of the guard biasing member 35 may continuously press the camming surfaces 53 of the lock ring 40 against the camming surfaces 55 of the inner annular wall 57. As a consequence, the lock ring 40 is continuously urged to rotate about the longitudinal axis A. However, the lock ring 40 may not rotate depending on the relative positions of various cooperating abutment structures included on the exterior of the lock ring 40 and the interior of the guard member 32. Depending on the axial position of the guard member 32, these cooperating abutment structures may come into and/or out of engagement with each other to allow the lock ring 40 to rotate. In some embodiments, the lock ring 40 may rotate into a final rotational position upon the guard member 32 moving from the retracted position to the extended position. In the final rotation position, a distally facing surface of one or more of the abutment structures included on the lock ring 40 may be rotationally aligned with and arranged in opposition to a proximally facing surface of one or more of the counterpart abutment structures included on the guard member 32. As a consequence, any subsequent movement of the guard member 32 in the proximal direction may be prevented by the distally surface(s) of the abutment structure(s) included on the lock ring 40 engaging the proximally facing surface(s) of the abutment structure(s) included on the guard member 32.

The drug delivery device 10 may further include a drive mechanism 30 disposed partially or entirely within the housing 12. Generally, the drive mechanism 30 may be configured to store energy and, upon or in response to activation of the drive mechanism 30 by the user, release or output that energy to drive the plunger 26 to expel the drug 22 from the drug storage container 20 through the delivery member 16 into the patient. In the present embodiment, the drive mechanism 30 is configured to store mechanical potential energy; however, alternative embodiments of the drive mechanism 30 may be configured differently, for example, with the drive mechanism 30 storing electrical or chemical potential energy. Generally, upon activation of the drive mechanism 30, the drive mechanism 30 may convert the potential energy into kinetic energy for moving the plunger 26.

In the present embodiment, the drive mechanism 30 includes the plunger biasing member 50, a plunger biasing member seat 38, the releaser member 52, and a plunger guide 60. The plunger biasing member 50 may include a compression spring (e.g., a helical compression spring) which is initially retained in an energized state. In the energized state, the plunger biasing member 50 may be compressed such that its axial length is shorter than it would be in a natural or de-energized state. When released, the plunger biasing member 50 may try to expand to its natural axial length, and as a consequence, exert a biasing force pushing the plunger 26 in the distal direction.

The plunger biasing member 50 may be disposed at least partially within the plunger 26, and may have a distal end abutting against a proximally facing inner surface of the plunger 26 and/or may be fixedly attached to an inner surface of the plunger 26. So that the plunger biasing member 50 may be received within the plunger 26, an outer diameter or other dimension of the plunger biasing member 50 may be equal to or less than an inner diameter of the top ring 45 and/or equal to or less than an inner diameter of the hollow rod 46. In some embodiments, the distal end of the plunger biasing member 50 may abut against a proximally facing inner surface of the base 47 of the plunger 26. Furthermore, a proximal end of the plunger biasing member 50 may abut against a distally facing surface of the plunger biasing member seat 38. The plunger biasing member seat 38 may be fixedly attached to the rear housing 27 such that the plunger biasing member seat 38 provides a stationary surface for the plunger biasing member 50 to push off of. So configured, the plunger biasing member 50, when released from the energized state, may expand in length with distal end of the plunger biasing member 50 moving in the distal direction away from the stationary proximal end of the plunger biasing member 50. This motion may push the plunger 26 is the distal direction, which, in turn, may push the stopper 24 in the distal direction to expel the drug 22 from the drug storage container 20 into the delivery member 16 and thereafter into the patient.

The plunger guide 60 may be fixedly attached to the rear housing 27 such that the plunger guide 60 is immovable relative to the rear housing 27. The plunger guide 60 may have a hollow and generally cylindrical or tubular shape, and may be centered about the longitudinal axis A. An outer diameter or other outer dimension of a proximal end of the plunger guide 60 may be larger than an outer diameter or other outer dimension of a distal end of the plunger guide 60. At least a portion of the distal end of the plunger guide 60 may be positioned radially between the plunger 26 and the releaser member 52. As such, the plunger 26 may be disposed at least partially within the distal end of the plunger guide 60, and the distal end of the plunger guide 60 may be disposed at least partially within the releaser member 52, as illustrated in FIG. 2.

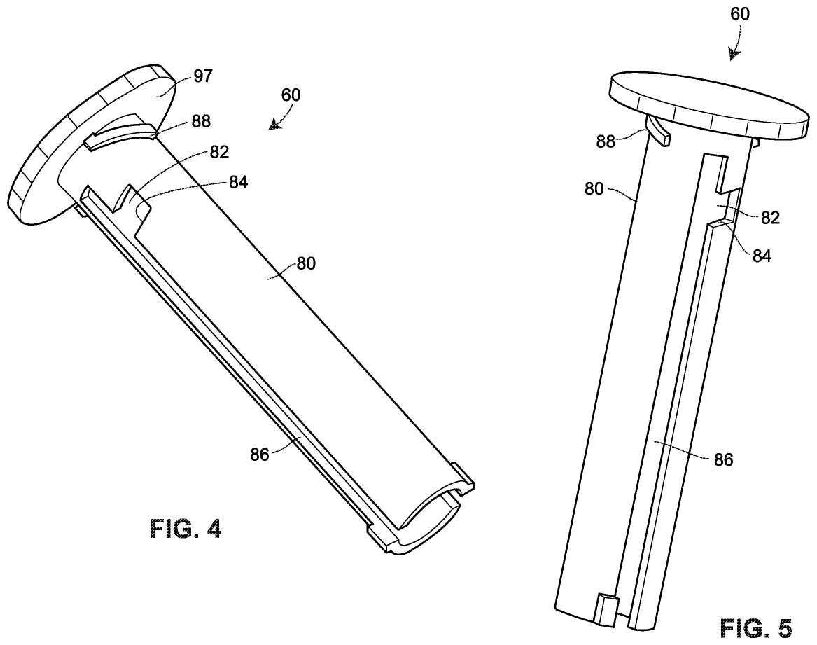

Referring to FIGS. 4, 5, and 8, the distal end of the plunger guide 60 may include an annular wall 80 formed with various surfaces and openings for interacting with and controlling movement of the plunger 26 and the releaser member 52. More particularly, a first opening 82 may be formed in the annular wall 80 and may be sized to receive one of the projections 48 extending outwardly from the top ring 45 of the plunger 26. The annular wall 80 may include a proximally facing camming surface 84 that defines a portion of the periphery of the first opening 82. The camming surface 84 may be sloped downwardly at angle relative to, or is otherwise non-parallel to, an imaginary plane perpendicular to the longitudinal axis A. In the pre-delivery state, the proximally facing camming surface 84 of the plunger guide 60 may be in contact with the distally facing camming surface 49 of the top ring 45 of the plunger 26. Here, the biasing force of the plunger biasing member 50 may press the distally facing camming surface 49 of the top ring 45 against the proximally facing camming surface 84 of the plunger guide 60. As a consequence, the distally facing camming surface 49 of the top ring 45 may be urged to slide along the proximally facing camming surface 84 of the plunger guide 60, generally following a spiral-like path. If permitted, this sliding motion may result in rotation, as well as linear translation, of the plunger 26 relative to the stationary plunger guide 60. Accordingly, the plunger guide 60 may function as a cam and the top ring 45 as a cam follower. In the pre-delivery state, any rotation of the plunger 26 relative to the plunger guide 60 may be prevented by engagement between the projection 48 and the releaser member 52, as described below. In the absence of sliding motion between the distally facing camming surface 49 of the top ring 45 and the proximally facing camming surface 84 of the plunger guide 60, the annular wall 80 of the plunger guide 60 acts to prevent linear translation of the plunger 26 in the distal direction. Thus, the plunger guide 60 may assist with retaining the plunger biasing member 50 in the energized state prior to retraction of the guard member 32. In some embodiments, an opening similar to the first opening 82 may be formed on the opposite side of the plunger guide 60, and may be configured to receive a different one of the projections 48 of the top ring 45.

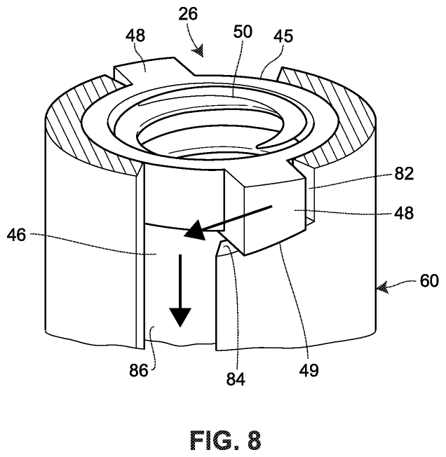

With continued reference to FIGS. 4, 5, and 8, a second opening 86 may be formed in the annular wall 80 of the plunger guide 60 and may be at least partially arranged distal to the first opening 86. As illustrated in FIGS. 4 and 5, the second opening 86 generally takes the form of a longitudinal slot that is parallel to the longitudinal axis A. The second opening 86 may be sized to receive one of the projections 48 of the top ring 45 and may permit the projection 48 to slide through the second opening 86 linearly in the distal direction. After the projection 48 has rotated beyond an end of the camming surface 84, the projection 48 may be received in the second opening 86 and subsequently translate linearly in the distal direction through the second opening 86 without further rotation of the projection 48 relative to the plunger guide 60, as depicted in FIG. 8. In some embodiments, an opening similar to the second opening 86 may be formed on the opposite side of the plunger guide 60, and may be configured to receive a different one of the projections 48 of the top ring 45.

The annular wall 80 of the plunger guide 60 may further include a distally facing camming surface 88. As depicted in FIGS. 4 and 5, the distally facing camming surface 88 may be part of a spiral-like projection extending outwardly from a remainder of the annular wall 80. The distally facing camming surface 88 may be sloped upwardly at angle relative to, or is otherwise non-parallel to, an imaginary plane perpendicular to the longitudinal axis A. As described below in more detail, a biasing force of the guard biasing member 35 may press a proximally facing camming surface of the releaser member 52 against the distally facing camming surface 88 of the plunger guide 60. As a consequence, the proximally facing camming surface of the releaser member 52 may be biased to slide along the distally facing camming surface 88 of the plunger guide 60, generally following a spiral-like path. If permitted, this sliding motion may result in rotation, as well as linear translation, of the releaser member 52 relative to the stationary plunger guide 60. Accordingly, the plunger guide 60 may function as a cam and the releaser member 52 as a cam follower. In some embodiments, a distally facing camming surface similar to the distally facing camming surface 88 may be formed on the opposite side of the plunger guide 60, and may be configured to engage a different proximally facing camming surface on the releaser member 52.

The configuration of the releaser member 52 will now be described with reference to FIGS. 2, 3, 6, and 7. The releaser member 52 may have a hollow and generally cylindrical or tubular shape, and may be centered about the longitudinal axis A. As illustrated in FIG. 2, the releaser member 52 may be positioned in the radial direction between the distal end of the plunger guide 60 and a proximal end of the guard extension 37. Furthermore, the releaser member 52 may be arranged radially inwardly of the guard biasing member 35. Generally, the releaser member 52 is configured to operably couple the guard member 32 and the plunger 26 in an activation sequence and to generate an audible signal indicating the end of drug delivery. So configured, the releaser member 52 is exploited to perform two separate functions, and thus reduces the number of moving parts required by the drug delivery device 10.

The releaser member 52 may be configured to rotate relative to the housing 12 and/or translate linearly relative to the housing 12, depending on the stage of operation of the drug delivery device 10. Initial rotation of the releaser member 52 associated with activation may be powered by the plunger biasing member 50 and/or the guard biasing member 35; whereas later rotation of the releaser member 52 associated with generation of the end-of-dose signal may be powered solely by the guard biasing member 35. Any linear translation of the releaser member 52 without rotation may be powered solely by the guard biasing member 35. In some embodiments, the releaser member 52 may translate linearly only in the proximal direction; however, alternative embodiments may permit linear translation of the releaser member 52 in both the proximal and distal directions.

The releaser member 52 may possess an annular wall 90 having a distal end and a proximal end. Generally, the distal end of the annular wall 90 is configured to assist with activating the drive mechanism 30, and the proximal end of the annular wall 90 is configured for generating the audible end-of-dose signal. As depicted in FIG. 2, a distally facing ledge or surface 91 formed on an outer portion of the annular wall 90 may abut against the proximal end of the guard biasing member 35. As such, the guard biasing member 35 may exert a biasing force on the releaser member 52 urging the releaser member 52 in the proximal direction.

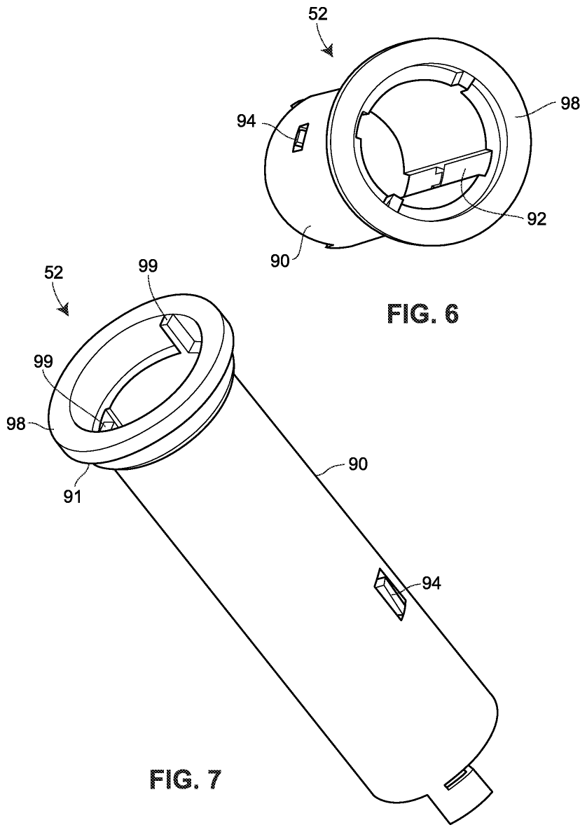

Referring to FIG. 6, a recess 92 may be formed in an inner portion of the annular wall 90 of the releaser member 52. In the present embodiment, the recess 92 takes the form of a groove formed in an inner surface of the annular wall 90. In other embodiments, the recess 92 may take the form of a through hole, opening, or slot extending between the inner and outer surfaces of the annular wall 90. The recess 92 may be arranged such that its length or longest dimension is parallel to the longitudinal axis A. Furthermore, the recess 92 may be sized to matingly or snugly receive one of the projections 48 of the top ring 45. The recess 92 may be configured to permit the projection 48 to slide linearly parallel to the longitudinal axis A relative to the releaser member 52, but prevent the projection 48 from rotating about the longitudinal axis A relative to the releaser member 52. This may be achieved by forming the recess 92 with a width that is slightly larger than a width of the projection 48, such that there is little play between the recess 92 and the projection 48 in the rotational direction. Due to the mating engagement between the projection 48 and the recess 92, the releaser member 52 and the plunger 26 may be rotationally locked to each other. As such, the releaser member 52 may rotate jointly together with the plunger 26 when the projection 48 is received within the recess 92; and when the projection 48 is not received within the recess 92, the releaser member 52 may be able to rotate independently of the plunger 26. In some embodiments, a recess similar to the recess 92 may be formed on the opposite side of the releaser member 52, and may be configured to receive the a different one of the projections 48 of the top ring 45.

An ability of the releaser member 52 to rotate about the longitudinal axis A may be regulated by an interaction between an outer portion of the annular wall 90 of the releaser member 52 and an inner portion of the guard extension 37. More particularly, the biasing force of the plunger biasing member 50 may continuously press the camming surface 49 of the projection 48 against the camming surface 84 of the plunger guide 90, thereby urging the projection 48 to rotate about the longitudinal axis A. Because the projection 48 is matingly received within the recess 92, the releaser member 52 may also be urged to rotate under the biasing force of the plunger biasing member 50. In addition, in some embodiments the releaser member 52 may be urged to rotate by the biasing force of the guard biasing member 35 via a camming arrangement between the proximal end of the releaser member 52 and plunger guide 60. Despite these biasing forces, in the pre-delivery state, the releaser member 52 is prevented from rotating by various cooperating abutment structures included on the outer portion of the annular wall 90 of the releaser member 52 and the inner portion of the guard extension 37. Depending on the relative axial positions of these abutment structures, the abutment structures may engage one another to prevent the releaser member 52 from rotating relative to the guard extension 37 or disengage from one another to allow the releaser member 52 to rotate relative to the guard extension 37. In the present embodiment, these cooperating abutment structures may take the form of: one or more projections 94 extending outwardly from the releaser member 52 and one or more corresponding projections 96 extending inwardly from the guard extension 37, which slidably engage one another to permit relative movement in a linear direction along the longitudinal axis A and contemporaneously abuttingly engage one another to prevent relative rotational movement about the longitudinal axis A. In certain alternative embodiments, the cooperating abutment structures may take the form of: one or more recesses formed in an outer surface of the releaser member 52 and one or more corresponding projections extending inwardly from the guard extension 37, which slidably engage one another to permit relative movement in a linear direction along the longitudinal axis A and contemporaneously abuttingly engage one another to prevent relative rotational movement about the longitudinal axis A. In certain other alternative embodiments, these cooperating abutment structures may take the form of: one or more projections extending outwardly from the releaser member 52 and one or more corresponding grooves formed in an inner surface of the guard extension 37, which slidably engage one another to permit relative movement in a linear direction along the longitudinal axis A and contemporaneously abuttingly engage one another to prevent relative rotational movement about the longitudinal axis A.

As described above, the guard extension 37 is prevented from rotating about the longitudinal axis A as a consequence of its coupling to the housing 12. This has the effect of preventing rotation of the releaser member 52 about the longitudinal axis A when the projections 94 on the outer portion of the releaser member 52 engage the projections 96 on the inner portion of the guard extension 37. If the releaser member 52 is unable rotate, the projection 48 received in the recess 92 formed in the inner surface of the releaser member 52 is also unable to rotate. If the projection 48 cannot rotate, then it cannot slide out of the first opening 82 and into the second opening 86 in the plunger guide 60. If the projection 48 cannot move in this manner, then plunger 26 also cannot move. If the plunger 26 cannot move, the plunger biasing member 50 cannot expand and de-energize. Thus, the releaser member 52 retains the plunger biasing member 50 in the energized state until the guard extension 37 moves to an axial position where the cooperating abutment structures on the outer portion of the releaser member 52 and the abutment structures on the inner portion of the guard extension 37 disengage from each and thereby permit the releaser member 52 to rotate relative to the guard extension 37.

In addition to this retaining function, the releaser member 52 may also be used to generate an audible signal indicating to the user that drug delivery or dosing is complete, although it is not required for the releaser member 52 to have this indicator function. In the present embodiment, a proximal end of the releaser member 52 defines the indicator. Thus, in the present embodiment, the indicator and the releaser member 52 are the same component. In alternative embodiments, the indicator may be defined by a structure that is separate from but rigidly attached to the releaser member 52.

Initially, a gap may exist between a proximally facing end surface 97 of the releaser member 52 and a distally facing abutment surface 98 of the proximal end of the plunger guide 60. To generate the audible signal, the releaser member 52 may be driven in the proximal direction by the guard biasing member 35 to close this gap and thus cause the proximally facing end surface 97 of the releaser member 52 to impact or strike the distally facing abutment surface 98 of the proximal end of the plunger guide 60. This impact may generate a click or slap sound, or any other suitable audible signal that is perceptible to the user. The audible signal may be generated simultaneously, or substantially simultaneously, with the stopper 24 reaching the end-of-dose position. Accordingly, the audible signal may signify to the user that drug delivery or dosing is complete. In some embodiments, the user may be informed of the significance of the audible signal by way of instructions provided with the drug delivery device 10. In some embodiments, these instructions may take the form of an IFU pamphlet packaged together with the drug delivery device 10. In some embodiments, the user may obtain additional confirmation that drug delivery is complete by watching movement of the stopper 24 and/or plunger 26 through the window 17. In some embodiments, the audible signal may be accompanied by a vibration or other tactile feedback produced as a result of the releaser member 52 striking the plunger guide 60.

In some embodiments, movement of the releaser member 52 to create the audible signal may involve both rotation of the releaser member 52 about the longitudinal axis A and linear translation of the releaser member 52 in the proximal direction. This may be achieved by a camming arrangement between the releaser member 52 and the plunger guide 60. In the present embodiment, the proximal end of the releaser member 52 includes a proximally facing camming surface 99 which slidably engages the distally facing camming surface 88 on the annular wall 80 of the plunger guide 60. A biasing force of the guard biasing member 35 may press the proximally facing camming surface 99 of the releaser member 52 against the distally facing camming surface 88 of the plunger guide 60. As a consequence, the proximally facing camming surface 99 of the releaser member 52 may be urged to slide along the distally facing camming surface 88 of the plunger guide 60, generally following a spiral-like path. If permitted, this sliding motion may result in rotation, as well as linear translation, of the releaser member 52 relative to the stationary plunger guide 60. Accordingly, the plunger guide 60 may function as a cam and the releaser member 52 as a cam follower. In some embodiments, a proximally facing camming surface similar to the proximally facing camming surface 99 may be formed on the opposite side of the releaser member 52, and may be configured to engage a different distally facing camming surface on the plunger guide 60.

Though the guard biasing member 35 may continuously urge the proximally facing camming surface 99 of the releaser member 52 to slide along the distally facing camming surface 88 of the plunger guide 60, such movement may be limited by the interaction between the projection 48 of the plunger 26 and the recess 92 formed in the releaser member 52. More particularly, when the projection 48 is received in the recess 92 and thus the plunger 26 and the releaser member 52 are configured to rotate jointly, rotation of the plunger 26 may allow for the proximally facing camming surface 99 of the releaser member 52 to slide along the distally facing camming surface 88 of the plunger guide 60, which, in turn, results in rotation of the releaser member 52 about the longitudinal axis A and linear translation of the releaser member 52 in the proximal direction. Conversely, when the projection 48 is received in the recess 92 and the projection 48 is unable to rotate, e.g., because the projection 48 is received in the second opening 86 formed in the plunger guide 60, then the proximally facing camming surface 99 of the releaser member 52 may not slide along the distally facing camming surface 88 of the plunger guide 60. As described below, when the stopper 24 arrives in the end-of-dose position, the projection 48 may slide out of a distal end of the recess 92. As a consequence, the releaser member 52 may be free to rotate about the longitudinal axis A. This allows the guard biasing member 35 to push the proximally facing camming surface 99 of the releaser member 52 to slide along the distally facing camming surface 88 of the plunger guide 60, which, in turn, closes the gap between the proximally facing end surface 97 of the releaser member 52 and the distally facing abutment surface 98 of the proximal end of the plunger guide 60 and culminates with the proximally facing end surface 97 striking or otherwise coming into contact with the distally facing abutment surface 98 to generate the audible signal indicative of the end of drug delivery.

While the foregoing embodiments utilize the guard biasing member 35 to provide the actuation energy needed generating the end-of-dose signal, alternative embodiments may utilize a biasing member that is separate from guard biasing member 35 for this purpose. In certain such embodiments, this additional biasing member may have a distal end fixed relative to the housing 12 and a proximal end abutting against a distally facing surface of the releaser member 52. As such, the biasing member may push off of the housing 12 to exert a biasing force in the proximal direction against the releaser member 52. Furthermore, this biasing member may operate independently of the plunger biasing member 50 and the guard biasing member 35.

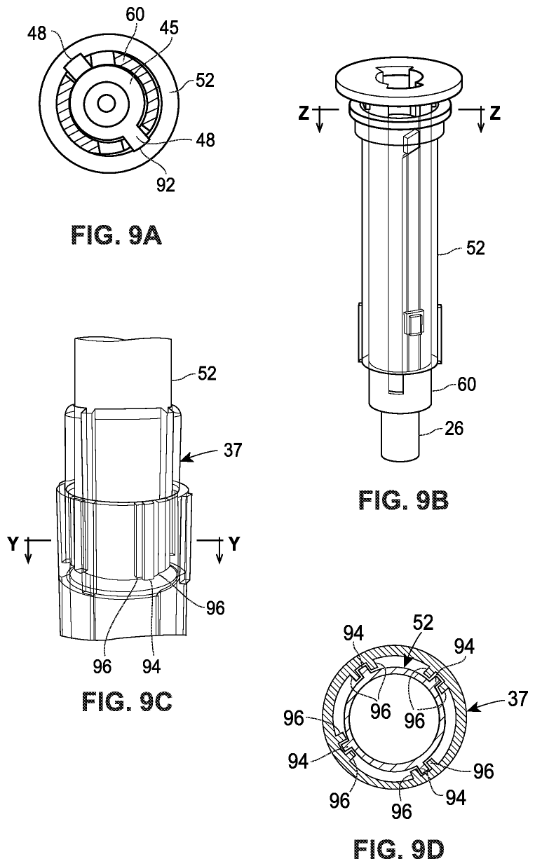

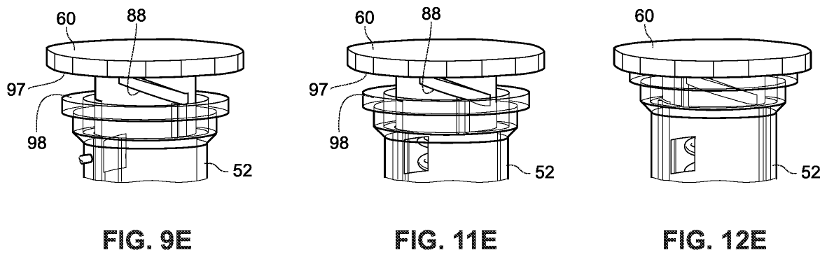

Having described the general configuration of the drug delivery device 10, a method of using the drug delivery device 10 to perform an injection will now be described with reference to FIGS. 9A-12E. As a preliminary step, the user may remove the drug delivery device 10 from any secondary packaging, such as a plastic bag and/or cardboard box. Also, as a preliminary step, the user may prepare the injection site, e.g., by rubbing the patient's skin with an alcohol wipe. Next, the user may pull and detach the removable cap 19 from the front housing 25. As a result of this motion, the gripper 13 may pull and detach the sterile barrier 21 from the drug storage container 20. This may uncover the insertion end 28 of the delivery member 16. Nevertheless, the insertion end 28 of the delivery member 16 will remain surrounded by the guard member 32 at this stage because the guard member 32 is arranged in the extended position. Next, the user may position the drug delivery device 10 over the injection site and then push the distal end of the guard member 32 against the injection site. The force applied by the user will overcome the biasing force of the guard biasing member 35 and the biasing force of the lock ring biasing member 51, thereby causing the guard member 32 to retract into the opening 14 moving from the extended position to the retracted position in the proximal direction. The delivery member 16 remains stationary relative to the housing 12 during the retracting movement of the guard member 32.

Movement of the guard member 32 from the extended position to the retracted position may cause several actions to occur. Because the delivery member 16 remains stationary relative to the housing 12 during retraction of the guard member 32, the insertion end 28 of the delivery member 16 is caused to extend through an opening in the distal end of the guard member 32, thereby piercing the patient's skin at the injection site and penetrating into the patient's subcutaneous tissue. In addition, retraction of the guard member 32 may also activate the drive mechanism 30 to expel the drug 22 from the drug storage container 20, as described below.

In the pre-delivery state prior to retraction of the needle guard 32, the plunger 26 and the releaser member 52 each may be arranged in a respective initial rotational position, as illustrated in FIGS. 9A-9E. Here, the projection 48 of the top ring 45 of the plunger 26 may extend through the first opening 82 in the plunger guide 60 and may be received in the recess 92 in the releaser member 52. Also, prior to needle guard retraction, the plunger biasing member 50 may be in an energized state. As a consequence, the plunger biasing member 50 may exert a distally directed biasing force on the plunger 26 which urges the distally facing camming surface 49 on the projection 48 to slide along the proximally facing camming surface 84 of the plunger guide 60. The resulting camming action may urge the plunger 26 to rotate in the clockwise direction in FIGS. 9A and 9E. In some embodiments, the plunger 26 may also be urged to rotate as a consequence of the guard biasing member 35 pushing the proximally facing camming surface 99 of the releaser member 52 against the distally facing camming surface 88 of the plunger guide 60. Despite these biasing force(s), neither the releaser member 52 nor the plunger 26 rotates in the pre-delivery state. This is because, as illustrated in FIG. 9D, each radially outwardly extending projection 94 on the outer portion of the releaser 50 abuts against a respective radially inwardly extending projection 96 on the inner portion of the guard extension 37. Because the guard biasing member 37 is rotationally fixed relative to the housing 12, the abutting engagement of the projections 94 and 96 prevents the releaser member 52 from rotating. This, in turn, prevents the plunger 26 from rotating due to the projection 48 being received within the recess 92 of the releaser member 52. The inability of the plunger 26 to rotate means that the projection 48 cannot slide out of the first opening 82 into the second opening 86, where the projection 48 would be free to translate linearly in the distal direction. Accordingly, the releaser member 52, the plunger guide 60, the guard extension 37, and the housing 12 work in conjunction with one another to retain the plunger biasing member 50 in the energized state prior to retraction of the guard member 32.

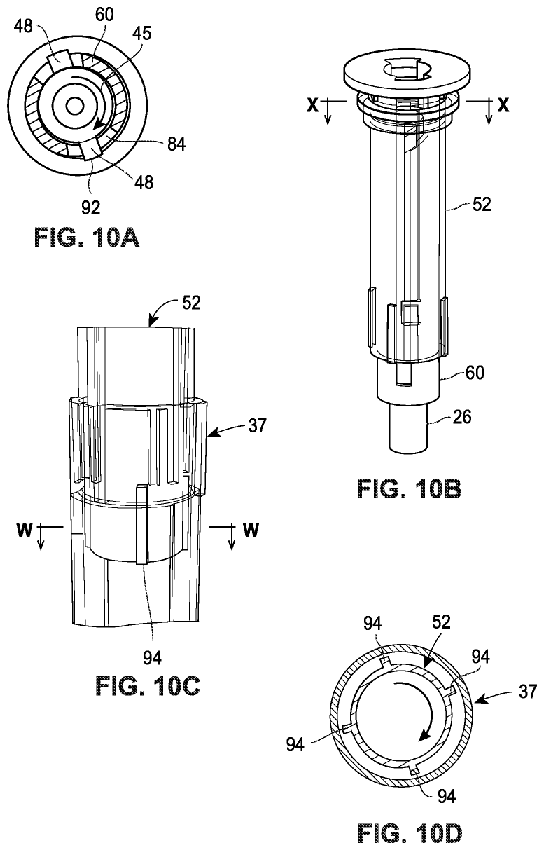

When the guard member 32 moves from the extended position to the retracted position, the guard member 32 may push the guard extension 37 in the proximal direction from the position shown in FIG. 9C to the position shown in FIG. 10C. During proximal movement of the guard extension 37, the projections 96 and 98 may slide past one another until finally the projections 96 and 98 are no longer in contact with one another (FIGS. 10C and 10D). When that occurs, the releaser member 52 may be free to rotate about the longitudinal axis A. Rotation of the releaser member 52 at the present stage is caused by the plunger biasing member 50 expanding and pushing the distally facing camming surface 49 on the projection 48 to slide along the proximally facing camming surface 84 of the plunger guide 60, as illustrated in FIGS. 10A and 10B. The resulting camming action causes the projection 48 to rotate, which, in turn, causes the releaser member 52 to jointly rotate due to the projection 48 being received within the recess 92. During this rotational movement, the plunger 26 translates linearly in the distal direction and the releaser member 52 translates linearly in the proximal direction. The distal translation of the plunger 26 is due to the downwardly sloping angle of the proximally facing camming surface 84 of the plunger guide 60, along which the projection 48 of the plunger 26 slides against under the distally directed biasing force of the plunger biasing member 50. The proximal translation of the releaser member 52 is due to the proximally directed biasing force exerted on the releaser member 52 by the guard biasing member 35. In some embodiments, during the proximal translation of the releaser member 52, the proximally facing camming surface 99 of the releaser member 52 may slide against the distally facing camming surface 88 of the plunger guide 60.

In some embodiments, the camming action between the distally facing camming surface 49 on the projection 48 and the proximally facing camming surface 84 of the plunger guide 60 may provide a damping effect. More particularly, a sliding friction between these two surfaces may be selected to slow initial expansion of the plunger biasing member 50. As a consequence, the velocity of the plunger 26 may be reduced during the initial expansion of the plunger biasing member 50, as compared to free uninhibited expansion of the plunger biasing member 50. The reduced velocity of the plunger 26 may cause the plunger 26 to strike the stopper 24 with less force, which reduces the chances of structural damage to the drug storage container 20 and/or facilitates a more comfortable injection for the user.

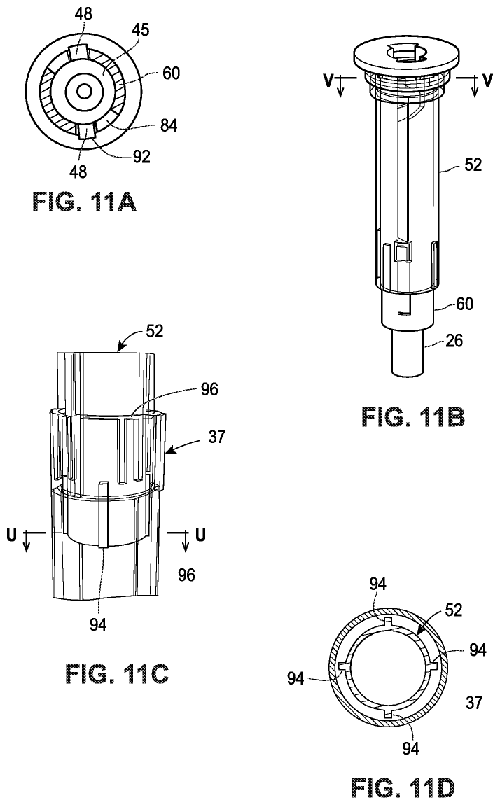

Joint rotation of the releaser member 52 and the plunger 26 may continue until the projection 48 slides off of the proximally facing camming surface 84 of the plunger guide 60, as seen in FIGS. 11A and 11B. Here, the projection 48 has moved out of the first opening 82 and into the second opening 86. The sidewalls of the second opening 86 may slidably and snugly receive the projection 48 such that there is little or no rotational play between them. Accordingly, the projection 48 and the rest of the plunger 26 may be prevented from rotating while the projection 48 is received in the second opening 86. Because the end of the projection 48 is still received within the recess 92 of the releaser member 52, the releaser member 52 may also be prevented at the present stage. The second opening 86 does not inhibit linear movement of the projection 48. Accordingly, the projection 48 along with the rest of the plunger 26 are driven by the expanding plunger biasing member 50 to translate linearly in the distal direction. As a consequence, the base 47 of the plunger 26 comes into contact with the stopper 24 and thereafter pushes the stopper 24 in the distal direction to expel the drug 22 from the drug storage container 20 through the delivery member 16 and out of the insertion end 28 into the patient's tissue.

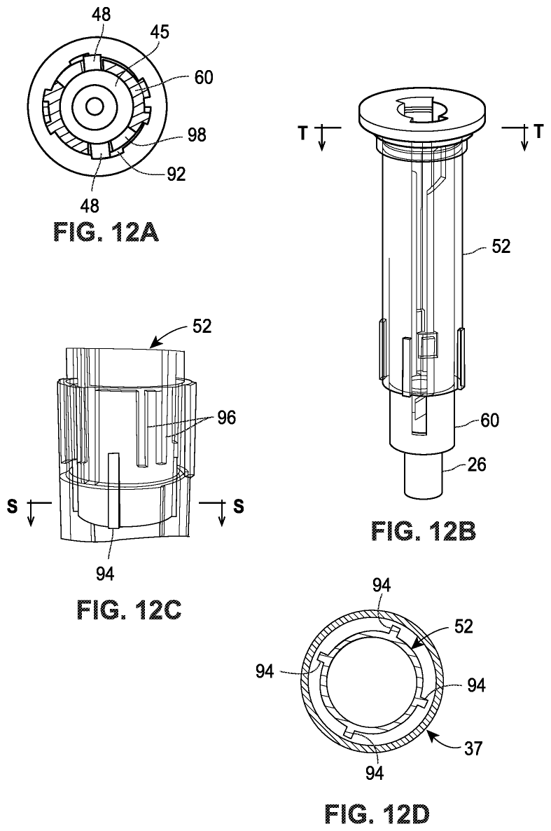

Drug delivery may carry on until the stopper 24 reaches the end-of-dose position. Here, the stopper 24 may abut against a proximally facing portion of the interior surface 15 of the wall of the drug storage container 20. As a result, the plunger 26 ceases moving in the distal direction. Simultaneous with, or substantially simultaneously with, the stopper 24 reaching the end-of-dose position, the projection 48 may slide out of the recess 92 on the releaser member 52, as shown in FIG. 12B. As a consequence, the releaser member 52 is now free to rotate about the longitudinal axis A. Rotation of the releaser member 52 at the present stage is caused by the guard biasing member 35 expanding and pushing the proximally facing camming surface 99 of the releaser member 52 to slide against the distally facing camming surface 88 of the plunger guide 60. The resulting camming action causes the releaser member 52 to rotate and translate linearly in the proximal direction. This motion may continue until the proximally facing end surface 97 of the releaser member 52 strikes the distally facing abutment surface 98 of the proximal end of the plunger guide 60 (FIG. 12E). This impact may generate the audible signal which indicates to the user that drug delivery is complete.

With some assurance that drug delivery is complete, the user may then lift the drug delivery deice 10 off of the injection site. With nothing to resist it, the guard biasing member 35 may push the guard member 32 from the retracted position to the extended position to cover the insertion end 28 of the delivery member 16. In some embodiments, this movement of the guard member 32 may cause the lock ring 40 to rotate to a position where it prevents subsequent retraction of the guard member 32.

From the foregoing, it can be seen that the present disclosure advantageously provides a streamlined design for a drug delivery device having automated features. Various mechanisms and components of the drug delivery device may interact with each other in synergistic ways so as to limit the number of moving parts required by the drug delivery device, thereby improving the reliability of the drug delivery device and saving costs, as well as providing other benefits and advantages.

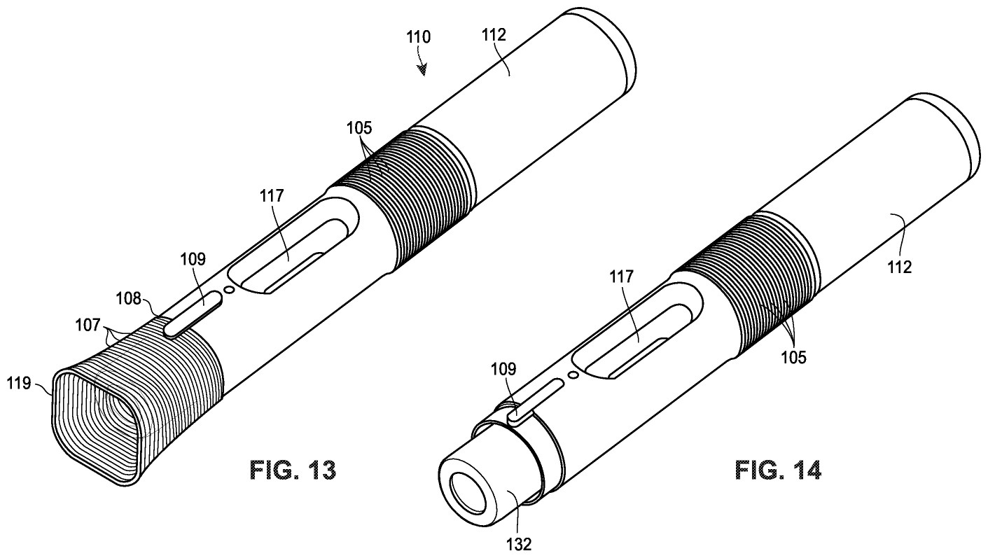

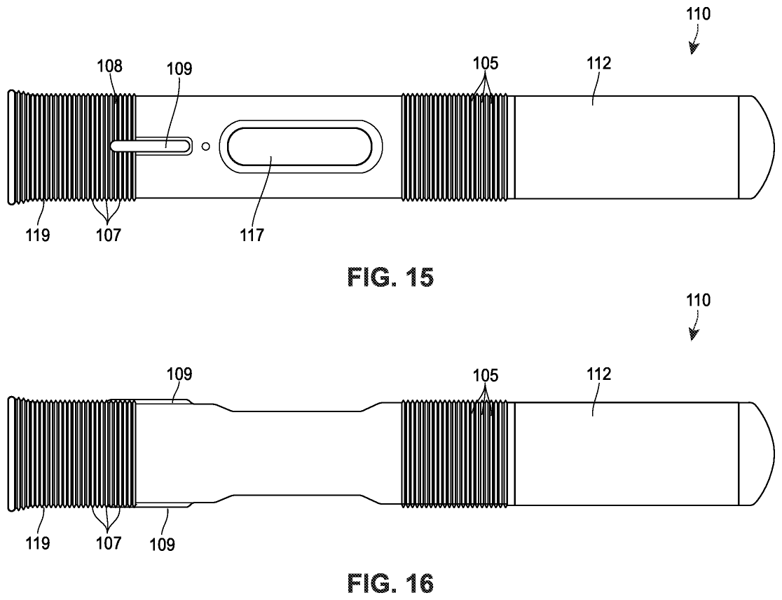

A variety of exterior form factors are possible for the drug delivery devices described herein depending on, for example, user and/or manufacturer needs and/or preferences. FIGS. 13-16 illustrate an embodiment of a drug delivery device 110 having the same or similar internal components as the drug delivery device 10 described above but having a different exterior form factor. Features of the drug delivery device 110 which are similar in function to those included in the drug delivery device 10 are assigned with same reference numeral except incremented by 100.

The drug delivery device 110 includes an outer casing or housing 112 having a generally elongate shape extending along a longitudinal axis. At most or all positions along the longitudinal axis the housing 112 may have a circular cross-section such that the housing 112 has a substantially cylindrical shape. A recess with a transparent or semi-transparent inspection window 117 may be positioned in a wall of the housing 112 to permit a user to view component(s) inside the drug delivery device 110, including, for example, a drug storage container. At a distal end of the housing 112, a removable cap 119 may cover an opening in the housing 112. The interior of the removable cap 119 may include a gripper configured to assist with removing a sterile barrier (e.g., a rigid needle shield (RNS), a flexible needle shield (FNS), etc.) from a delivery member such a needle when the removable cap 119 is removed from the housing 112, as described above. The housing 112 and the removable cap 119 may each have, respectively, a plurality of ribs 105 and 107 formed on their exterior surface to improve the user's ability to grip these components when pulling them apart. Each of the ribs may extend entirely or partially around the periphery of the housing 112 or the removable cap 119.

The circular cross-section of the housing 112 can make it prone to rolling across a surface when placed on its side. To inhibit or prevent such rolling, a portion or the entirety of the removable cap 119 may have a non-circular cross-section. In the embodiment illustrated in FIGS. 13-16, the removable cap 119 has a distal end with a non-circular cross-section and a proximal end with a circular cross-section. As such, the cross-section of the removable cap 119 gradually transitions from a circular cross-section to a non-circular cross-section when moving from the proximal end of the removable cap 119 to the distal end of the removable cap 119. In the illustrated embodiment, the non-circular cross-section of the distal end of the removable cap 119 generally takes the form of a square. In other embodiments, the non-circular cross-section may be rectangular, triangular, or any other polygonal or partially polygonal shape, so long one or more sides removable cap 119 are flat or substantially flat to inhibit or prevent rolling. Furthermore, the non-circular cross-section of the distal end of the removable cap 119 may gradually increase in size moving in the distal direction, such that the distal-most portion of the distal end of the removable cap 119 has a larger cross-sectional area than a proximal-most portion of the distal end of the removable cap 119. This configuration gives the distal end of the removable cap 119 a flared shape, which, in turn, may help a user grip and pull the removable cap 119 off of the housing 112.

In some embodiments, the housing 112 and the removable cap 119 may each include a respective anti-rotation feature. These anti-rotation features may engage each other to prevent or inhibit the removable cap 119 from rotating relative to the housing 112 when the removable cap 119 is in a storage position such as that illustrated in FIG. 13. In some embodiments, the anti-rotation feature of the housing 112 may be adjacent to and generally in-line with the anti-rotation feature of the removable cap 119 when the removable cap 119 is in the storage position. In the embodiment illustrated in FIGS. 13-16, the anti-rotation feature of the removable cap 119 is provided by an opening 108 formed in the tubular wall of the removable cap 119 at the proximal end of the removable cap 119; and the anti-rotation feature of the housing 112 is provided by an axial protrusion 109 extending in the distal direction from the distal end of the housing 112. The opening 108 may be sized to matingly receive an axial protrusion 109 when the removable cap 119 is in the storage position. As a consequence of this mating engagement, the removable cap 119 may be unable to rotate relative to the housing 112. This may be beneficial if a user attempts to twist the removable cap 119 when pulling the removable cap 119 off of the housing 112. In certain cases, rotation of the removable cap 119 may cause a sterile barrier such as an RNS or FNS to rotate, which, in turn, may cause a tip of a needle to core into a seal member within the RNS or FNS. Thus, having the axial protrusion 109 disposed within the opening 108 at least during the initial moments of cap removal may prevent coring of the needle. In alternative embodiments, the opening 108 may be formed in the wall of the housing 112 and the axial protrusion 109 may extend in the proximal direction from a proximal end of the removable cap 119.

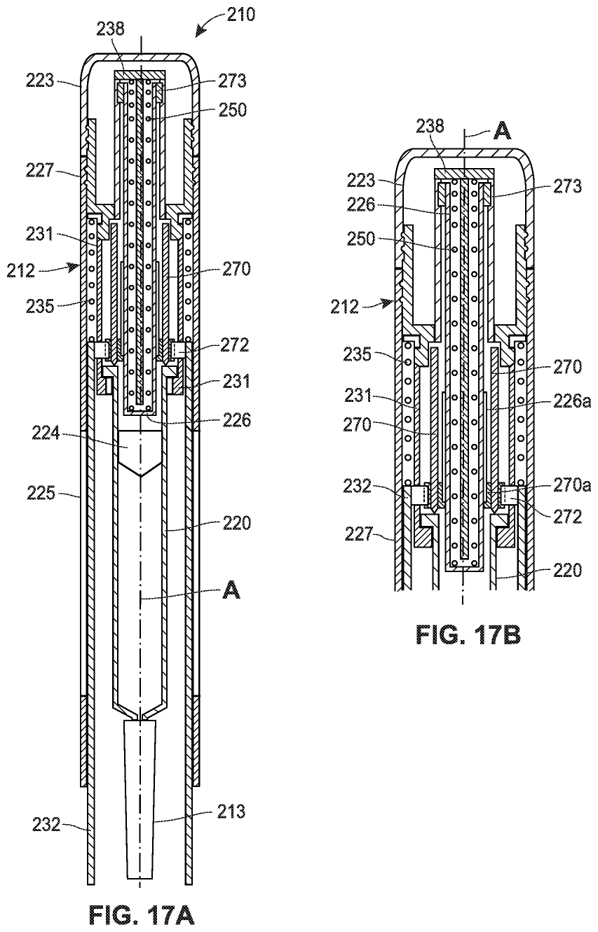

Turning to FIGS. 17A-21, various embodiments of a drug delivery device incorporating a brake member will now be described. Various elements of the drug delivery devices illustrated in FIGS. 17A-21 may be similar in function and/or structure to elements of the drug delivery device 10 described above in conjunction with FIGS. 1-12E. Such elements are assigned with the same reference numeral as used in FIGS. 1-12E, except incremented by 100 or a multiple thereof. Details of the structure and/or function that differentiate the embodiments illustrated in FIGS. 17A-21 from the embodiment in FIGS. 1-12E are the focus of the discussion below. Although they may not be illustrated in FIGS. 17A-21, components of the drug delivery device 10 or variants of these components may be included in the various drug delivery devices described in connection with FIGS. 17A-21 unless the design of the particular drug delivery device prevents the inclusion of these components or the variants thereof.

The inclusion of a brake member is advantageous at least in drug delivery devices where, in a pre-delivery or storage state, a distal end of a plunger is spaced from a proximal end of a stopper by a gap. As an example, FIG. 17A illustrates a drug delivery device 210 in a pre-delivery or storage state where a distal end of the plunger 226 is spaced from proximal end of the stopper 224 by a gap (e.g., an axial distance). The gap may be a consequence of, for example, the drug storage container being filled with a certain volume of a drug, design tolerances, and/or manufacturing considerations. Because of the gap, the plunger, upon the release of the plunger biasing member, may be allowed to accelerate to a significant velocity and strike the stopper with significant force. This, in turn, may create an impulse or shockwave which, in certain cases, may shatter or damage a wall of the drug storage container, which may be made of glass, and/or startle the user. Additionally, in embodiments where the plunger biasing member is a spring, the output force of the plunger biasing member may be greatest in the initial moments after its release. As a result, the plunger may attain significant velocity prior to striking the stopper.

The embodiments described below incorporate a brake member which is configured to resist movement of the plunger in the distal direction at least during a time period when the plunger is moving to close the initial gap between plunger and the stopper. As a result of the resistance provided by the brake member, the velocity of the plunger may be reduced during initial expansion of the plunger biasing member as compared a velocity of the plunger if the plunger biasing member was allowed to expand freely without impediment. The reduced velocity of the plunger has the effect limiting the amount of force with which the plunger strikes the stopper, which, in turn, reduces the possibility of structural damage to the drug storage container and furthermore may facilitate a more comfortable injection for the user or patient. In some embodiments, the brake member may cease resisting movement of the plunger simultaneously or nearly simultaneously with the plunger striking the stopper; whereas, in other embodiments, the brake member may continue resisting to plunger movement in the distal direction after the plunger strikes the stopper including, for example, throughout the entire plunger stroke. In some embodiments, the brake member may be operably (e.g., interactively) coupled to the plunger such that movement of the plunger in the distal direction causes the plunger and/or the brake member to rotate about a longitudinal axis of the drug storage container and/or a housing of the drug delivery device. The force needed to overcome the resting rotational inertia of and begin rotating the plunger and/or brake member may reduce the amount of force available for driving the plunger in the distal direction and thus may limit the velocity of the plunger in the distal direction. So configured, the brake member may operate like a damper in that the brake member dissipates kinetic energy associated with movement of the plunger in the distal direction. In some embodiments, the brake member may convert linear motion of the plunger into heat and/or other forms of energy in addition to rotational motion.

FIGS. 17A and 17B illustrate a drug delivery device 210 including a brake member 270 operably coupled to the plunger 226. The brake member 270 may surround at least a portion of the plunger 226 and may have an annular shape such as, for example, a ring, a hollow tube, or the like. In some embodiments, the annular shape of the brake member 270 may be centered along the longitudinal axis A. The operable coupling between the brake member 270 and the plunger 226 may be such that movement of the plunger 226 in the distal direction along the longitudinal axis A causes the brake member 270 to rotate. As an example, the brake member 270 may threadably engage the plunger 226 such that relative axial movement between the plunger 226 and the brake member 270 causes rotation of the brake member 270 about the longitudinal axis A. As a more specific example, the brake member 270 may have a threaded inner surface 270a which engages a threaded outer surface 226a of the plunger 226, as seen in FIG. 17B. By requiring the plunger 226 to rotate the brake member 270 as the plunger 226 moves in the distal direction, the brake member 270 may resist movement of the plunger 226 in the distal direction. In some embodiments, an axial length of the threaded inner surface 270a of the brake member 270 and/or the threaded outer surface 226a of the plunger 226 may be such that the brake member 270 resists distal movement of the plunger 226 during the entire or substantially the entire stroke of the plunger 226. In other embodiments, the axial length of the threaded inner surface 270a of the brake member 270 and/or the threaded outer surface 226a of the plunger 226 may be such that the brake member 270 resists distal movement of the plunger 226 during a limited portion of the stroke of the plunger 226 such as, for example, only during a portion of the stroke where the plunger 226 closes the gap between the plunger 226 and the stopper 224.

In order to prevent the plunger 226 from rotating about the longitudinal axis A as a result of its interaction with the brake member 270, a splined connection may be formed between the plunger 226 and the housing 212. While the splined connection may prevent rotation of the plunger 226, it may permit axial movement of the plunger 226. As an example, a spline 274 may be formed on the outer surface of the proximal end of the plunger 226 and may mate with a spline formed on an inner surface of the housing 212 or a component rotationally fixed to the housing 212.

In a pre-delivery or storage state, the brake member 270 may be prevented from rotating and as a consequence the plunger 226 due to its threaded coupling with the brake member 270 may prevented from moving in the distal direction under the biasing force of the plunger biasing member 250. As an example, the drug delivery device 210 may include a lock 272 which selectively prevents rotation of the brake member 270 relative to the plunger 226 and/or the housing 212. As a more specific example, the drug delivery device 210 may include a lock 272 having an initial position in which the lock 272 prevents the brake member 270 from rotating (as seen in FIGS. 17A and 17B) and a second position in which the lock 272 does not prevent the brake member 270 from rotating. In some embodiments, the lock 272 may be a rotary lock. The lock 272 may in some embodiments travel in the proximal direction in moving from the initial position to the second position. Additionally or alternatively, the lock 272 may deflect outwardly in the radial direction when moving from the initial position to the second position. In some embodiments, such deflection may be achieved by constructing the lock 272 of a resilient (e.g., elastic) material which, after a separate blocking component is removed, naturally returns to an original shape and/or bends as a result of a camming action between the lock 272 and the plunger 226 moving in the distal direction under the biasing force of the plunger biasing member 250.

In some embodiments, the lock 272 may be operably coupled to the guard member 232 such that moving the guard member 232 from the extended position to the retracted position causes the lock 272 to move from the initial position to the second position, thereby unlocking rotation of the brake member 270 and thus permitting axial expansion of the plunger biasing member 250 to drive the plunger 226 in the distal direction to expel the drug from the drug storage container 220.

According to some embodiments, the drug delivery device 210 may operate as follows. Initially (e.g., in the pre-delivery or storage state), the lock 272 may be arranged in its initial position such that the lock 272 prevents the brake member 270 from rotating. At this time the plunger biasing member 250 may urge the plunger 226 in the distal direction; however, the plunger 226 may be prevented from moving in the distal direction due to the threaded engagement between the plunger 226 and the currently rotationally-locked brake member 270. Subsequently, the user may press a distal end of the guard member 232 against the skin at an injection site. This may cause the guard member 232 to retract into the housing 212, moving from the extended position to the retract position. As a result of this movement, the guard member 232 may push the lock 272 in the proximal direction such that the lock 272 moves from the initial position to the second position. In the second position, the lock 272 may disengage from the brake member 270 such that the brake member 270 is free to rotate. The plunger biasing member 250 then begins to expand, pushing the plunger 226 in the distal direction to close the gap between the plunger 226 and stopper 224. Due to the threaded coupling between the plunger 226 and the brake member 270, distal translation of the plunger 226 causes the brake member 270 to rotate while the plunger 226 moves to close the gap between the plunger 226 and the stopper 224. Rotation of the brake member 270 absorbs a portion of the kinetic energy output by the plunger biasing member 250, leaving less kinetic energy for driving the plunger 226 in the distal direction. As a result, the velocity of the plunger 226 in the distal direction is less than if the brake member 270 was not included, at least at a moment in time when the distal end of the plunger 226 strikes the proximal end of the stopper 224. After contact with the stopper 224, the plunger biasing member 250 may push the plunger 226 in the distal direction, thereby causing the stopper 224 to move the drug out of the drug storage container 220 through the delivery member (e.g., a needle) and into the patient's body. The brake member 270 may continue rotate after the plunger 226 contacts the stopper 224 but this is not required.

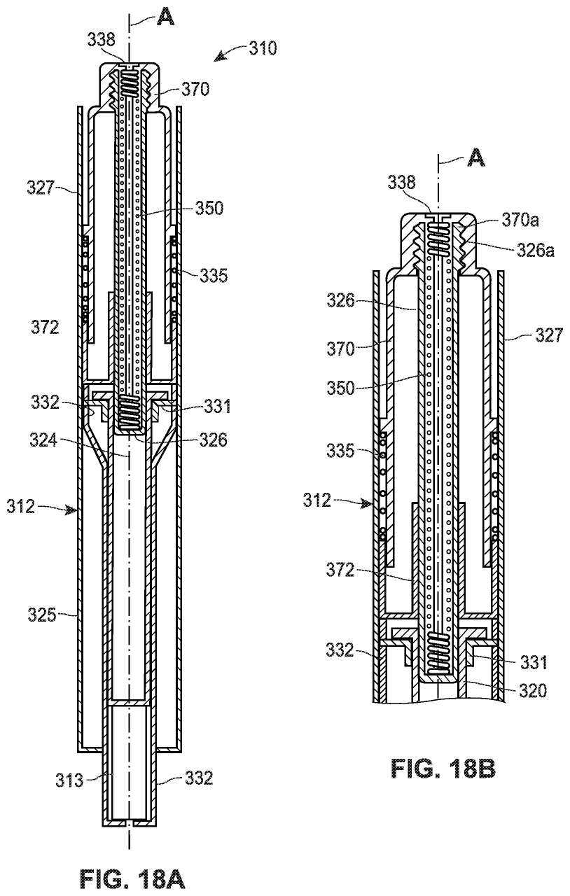

FIGS. 18A and 18B illustrate an embodiment of a drug delivery device 310 which has similarities in structure and/or function to the drug delivery device 210 in FIGS. 17A and 17B. Details of the structure and/or function that differentiate the drug delivery device 310 in FIGS. 18A and 18B from the drug delivery device 210 in FIGS. 17A and 17B are discussed below.

The drug delivery device 310 includes a plunger 326 and a brake member 370 operably coupled to each other such that the brake member 370 causes the plunger 326 to rotate when the plunger 326 moves in the distal direction. As an example, the brake member 370 may have a threaded inner surface 370a which threadably engages a threaded outer surface 326a at a proximal end of the plunger 326, as seen in FIG. 18B. The brake member 370 may be rotationally fixed to the housing 312 such that the brake member 370 is prevented from rotating about the longitudinal axis A. In some embodiments, the brake member 370 may be part of the housing 312, such as, for example, being the rear cover of the housing 312. Because the brake member 370 does not rotate, the threaded coupling between the brake member 370 and the plunger 326 causes the plunger 326 to rotate when the plunger 326 moves in the distal direction. The rotation of the plunger 326 absorbs a portion of the kinetic energy output by the plunger biasing member 350, leaving less kinetic energy for driving the plunger 326 in the distal direction. As a result, the velocity of the plunger 326 in the distal direction is less than if the brake member 370 was not included. After the plunger 326 has moved a certain distance in the distal direction, the threaded outer surface 326a of the plunger 326 may no longer contact the threaded inner surface 370a of the brake member 370. Once this occurs, the plunger 326 may cease rotating. In some embodiments, the axial length of the threaded inner surface 370a of the brake 370 may be equal to or substantially equal to the axial length of the initial gap between the distal end of the plunger 326 and the stopper 324. As a result, the plunger 326 may cease rotating simultaneously or nearly simultaneously with the plunger 326 striking the stopper 324.

In some embodiments, the plunger biasing member 350 may rotate jointly with the plunger 326. In such embodiments, a proximal end of the plunger biasing member seat 338, which may be in contact with a proximal end of the plunger biasing member 350, may be configured as a bearing. For example, the proximal end of the plunger biasing member seat 338 may be rotatably coupled to the brake member 370 and/or the rear housing 327 such that the plunger biasing member seat 338 is able to rotate relative to the brake member 370 and/or the rear housing 327. Accordingly, the plunger biasing member 350, the plunger 326, and the plunger biasing member seat 338 may rotate together in unison when the plunger 326 rotates as a result of the threaded coupling between the plunger 326 and the brake member 370.

The brake member 370 may be coupled to a proximal end of the guard biasing member 335. As an example, the proximal end of the guard biasing member 335 may be seated against the brake member 370, as seen in FIG. 18B. As a more specific example, the guarding biasing member 335 may surround a distal end of the brake member 370 and the guard biasing member 335 may have a proximal end that is seated against a flange extending radially outwardly from the brake member 370, as seen in FIG. 18B.

The drug delivery device 310 may further include a lock 370. The lock 370 may be similar to the lock 270 described above, except that the lock 370 prevents the plunger 326 from rotating in the pre-delivery or storage state. Without the ability to rotate, the plunger 326 may be prevented from moving in the distal direction due to the threaded coupling between the plunger 326 and the brake member 370. Accordingly, the lock 370 may prevent drug delivery until the lock 370 disengages from the plunger 326, which may occur in response to retraction of the guard member 332. The lock 370 may be disposed between the guard biasing member 335 and the guard member 332, as seen in FIG. 18B. The guard biasing member 335 may urge the lock 370 in the distal direction, and the lock 370 in turn may urge the guard member 332 toward the extended position.

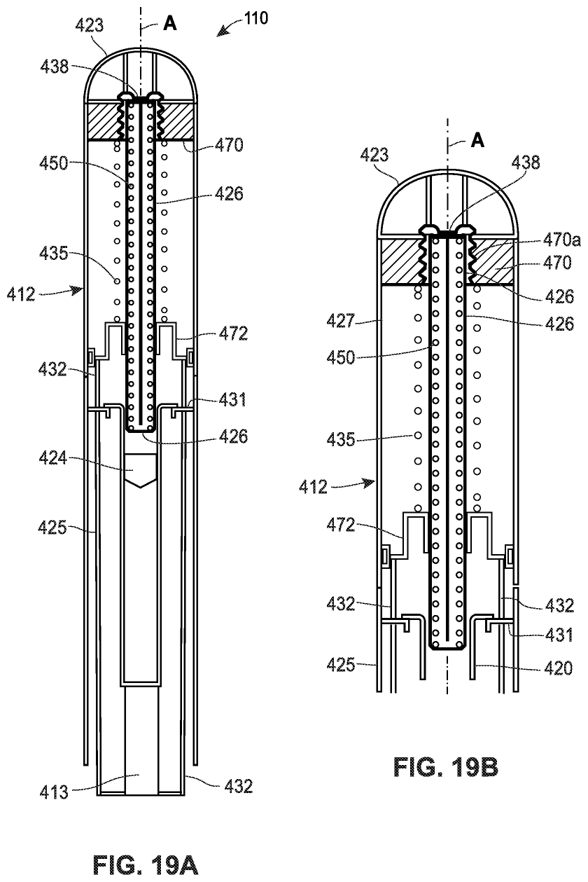

FIGS. 19A and 19B illustrate an embodiment of a drug delivery device 410 which has similarities in structure and/or function to the drug delivery device 310 in FIGS. 18A and 18B. Details of the structure and/or function that differentiate the drug delivery device 410 in FIGS. 19A and 19B from the drug delivery device 310 in FIGS. 18A and 18B are discussed below.

The drug delivery device 410 may include a brake member 470 that is part of the rear housing 427 of the drug delivery device 410. As an example, the brake member 470 may be defined by an annular flange extending radially inwardly from a proximal end of the rear housing 427, as seen in FIG. 19B. The inner surface of this flange may define the threaded inner surface 470a of the brake member 470.

The brake member 470 may be coupled to a proximal end of the guard biasing member 435. As an example, the proximal end of the guard biasing member 435 may be seated against a distally directed end surface of the brake member 470, as seen in FIG. 19B.

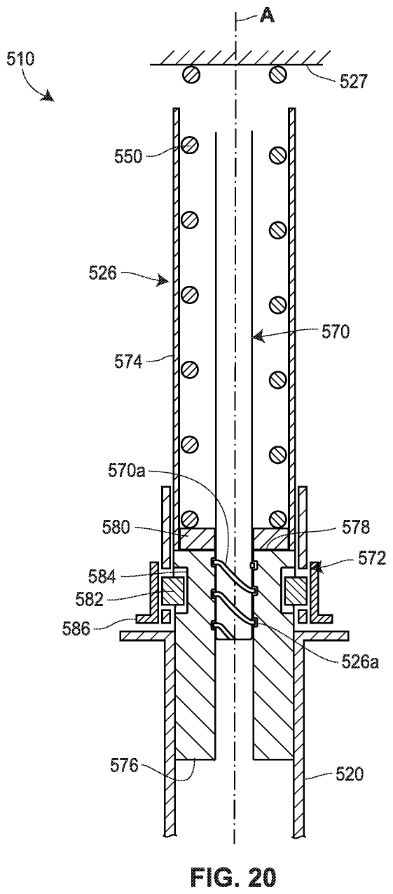

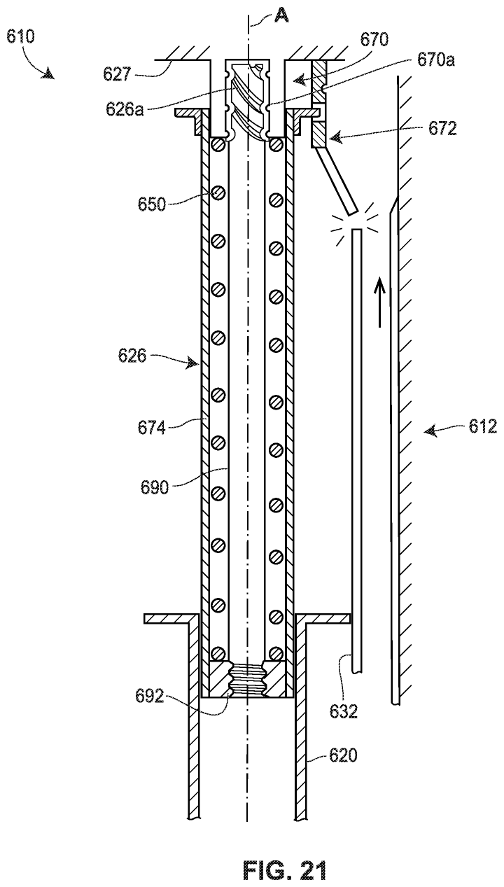

While the foregoing embodiments described in connection with FIGS. 17A-19B utilize a brake member which engages with an outer portion of the plunger, the embodiments described below in connection with FIGS. 20 and 21 utilize a brake member which engages with an inner portion of the plunger. Depending on the design of the drug delivery device, this configuration of the brake member can be advantageous. For example, in embodiments where the plunger is hollow and the plunger biasing member is disposed at least partially within the plunger, configuring the brake member so that it engages with an inner portion of the plunger may allow the plunger to be designed with a larger diameter than may otherwise be possible. This, in turn, may allow for the use of a spring with larger diameter for the plunger biasing member. A larger diameter spring may output more force when driving the plunger to expel the drug, which is beneficial, for example, for delivering viscous drugs such as certain biologic drugs. Furthermore, a larger diameter of the spring may allow one to decrease the axial length of the spring without compromising the force output by the spring. A shorter axial length of the spring may facilitate a smaller, more compact design of the drug delivery device, which may be desirable for handling, transport, and/or storage purposes or other purposes.

FIG. 20 illustrates an embodiment of a drug delivery device 510 which has similarities in structure and/or function to the drug delivery device 410 in FIGS. 19A and 19B. Details of the structure and/or function that differentiate the drug delivery device 510 in FIG. 20 from the drug delivery device 410 in FIGS. 19A and 19B are discussed below.

The drug delivery device 500 may include a plunger 526 having generally hollow tubular shape which defines an axial chamber. In some embodiments, the axial chamber may extend through the entire plunger 526 such that a proximal end and a distal end of the plunger 526 each has an opening communicating with an interior space of the plunger 526; whereas, in other embodiments, the axial chamber may extend through a limited portion of the plunger 526 such that, for example, the distal end of the plunger 526 is closed.

An interior of the plunger 526 may be configured to house the plunger biasing member 550 and additionally interface with the brake member 570. As an example, the proximal end of the plunger 526 may define a guide 574 and the distal end of the plunger 526 may define a nut 576. As illustrated in FIG. 20, the guide 574 may have an inner diameter or other dimension that is larger than an inner diameter or other dimension of the nut 576. The plunger biasing member 550 may be at least partially disposed within the guide 574 and have a distal end that is seated and/or pushing against a proximally-facing surface 578 of the nut 576. An annular bearing 580 may be disposed between the distal end of the plunger biasing member 550 and the proximally-facing surface 578 of the nut 576 and may be configured to allow the plunger 526 to rotate relative to the plunger biasing member 550 during axial expansion of the plunger biasing member 550. In some embodiments, the annular bearing 580 may include a washer. In other embodiments, the annular bearing 580 may be omitted and the distal end of the plunger biasing member 550 may be in direct contact with the proximally-facing surface 578 of the nut 576. The nut 576 may have a threaded inner surface 526a which, as described in more detail below, threadably engages a threaded outer surface 570a of the brake member 570. In the embodiment illustrated in FIG. 20, the distal end of the nut 576 has an opening. In some embodiments, a plug may be disposed in this opening and may have a distal end configured to be received in recess formed in the proximal end of the stopper.

In some embodiments, the guide 574 and the nut 576 may be integrally formed to define a single, one-piece structure. In other embodiments, the guide 574 and the nut 756 may be separate structures which are fixed to each other. In certain such embodiments, the guide 574 and nut 576 may be made of different materials. For example, the guide 574 may be made of metal and the nut 576 may be made of plastic, or vice versa. In some embodiments, the entirety of the plunger 526, including the guide 574 and the nut 576, may be made of a single material such as metal, plastic, or any other suitable material.

The brake member 570 may be operably coupled to the nut 576 such that the brake member 570 resists movement of the plunger 526 in the distal direction during at least an initial portion of the stroke of the plunger 526. As an example, the brake member 570 may include a rod or other elongated member having a proximal end fixed to the rear housing 527 and a distal end threadably engaged with the nut 576. As a more specific example, the brake member 570 may extend through the axial chamber of the plunger 526 and have a distal end including a threaded outer surface 570a which threadably engages the threaded inner surface 526a of the nut 576, as seen in FIG. 20. As a result of the threaded coupling between the brake member 570 and the nut 576 of the plunger 526, the brake member 570 may cause the plunger 526 to rotate about the longitudinal axis A when the plunger 526 moves in the distal direction. By requiring the plunger 526 to rotate, the brake member 570 may resist movement of the plunger 526 in the distal direction and thus reduce the velocity of the plunger 526 in the distal direction as compared to if the brake member 570 was omitted.

In the pre-delivery or storage state (seen in FIG. 20), the plunger 526 may be prevented from moving in the distal direction under the biasing force of the plunger biasing member 550. As an example, the drug delivery device 510 may include a lock 572 which has an initial position (FIG. 20) in which the lock 572 prevents movement of the plunger 526 in the distal direction and a second position in which the lock 572 does not prevent movement of the plunger 526 in the distal direction. As a more specific example, the lock 572 may include one or more generally radially inwardly extending arms 582 which, in the pre-delivery or storage state, are received in one or more corresponding recesses 584 formed in the outer surface of the plunger 526. The one or more radially inwardly extending arms 582 may be prevented from deflecting radially outwardly by a trigger ring 586 which surrounds the radially inwardly extending arms 582 in the pre-delivery or storage state. The trigger ring 586 may be operably coupled to a guard member (e.g., the guard member 32) such that upon retraction of the guard member in the proximal direction the trigger ring 586 also moves in the proximal direction and as a result no longer prevents the radially inwardly extending arms 582 from deflecting outwardly. In some embodiments, such deflection may be achieved by constructing the radially inwardly extending arms 582 of a resilient (e.g., elastic) material which, after the trigger ring 586 is moved out of the blocking position shown in FIG. 20, naturally return to an original shape and/or bend as a result of a camming action between the radially inwardly extending arms 582 and the corresponding recesses 584 of the plunger 526 as the plunger 526 is moved in the distal direction by the plunger biasing member 550. In some embodiments, the trigger ring 586 may be part of the guard member; whereas, in other embodiments, the trigger ring 586 may be separate from the guard member.

According to some embodiments, the drug delivery device 510 may operate as follows. Initially (e.g., in the pre-delivery or storage state), the lock 572 may be arranged in its initial position such that the radially inwardly extending arms 582 are received in respective recesses 584 in the plunger 526 and are prevented from deflecting radially outwardly by the trigger ring 586, as shown in FIG. 20. In this configuration, the lock 572 may prevent the plunger 526 from moving in the distal direction under the biasing force of the plunger biasing member 550. Subsequently, the user may press a distal end of the guard member against the skin at an injection site. This may cause the guard member to retract into the housing in the proximal direction and as a result push the trigger ring 586 in the proximal direction out of its initial blocking position. The radially inwardly extending arms 582 are therefore able to deflect radially outwardly, out of their respective recesses 584. Subsequently or simultaneously, the plunger 526 may begin to translate in the distal direction under the biasing force of the plunger biasing member 550. Due to the threaded coupling between the plunger 526 and the brake member 570, distal translation of the plunger 526 may cause the plunger 526 to rotate. As a result of this rotation, the plunger 526 may move in the distal direction with less velocity than if the plunger 526 was not required to rotate as result of its interaction with the brake member 570. Rotation of the plunger 526 may continue for as long as the threaded outer surface 526a of the plunger 526 remains in contact with the threaded inner surface 570a of the lock 572. In some embodiments, rotation of the plunger 526 may cease simultaneously or nearly simultaneously with the plunger 526 striking a stopper disposed in the drug delivery container 520.

In the embodiment illustrated in FIG. 20, a proximal end of the nut 576 is fixed to a distal end of guide 574. In alternative embodiments, a distal end of the nut 576 may be fixed to the distal end of the guide 574 such that the nut 576, along with the plunger biasing member 550, is disposed within an interior space of the guide 574. This may reduce the overall axial length of the plunger 526. In such alternative embodiments, the distal end of the guide 574 may include a transverse wall that is perpendicular or substantially perpendicular to the longitudinal axis A. In addition to being fixed to the distal end of the nut 576, the transverse wall may define a seat for the distal end of the plunger biasing member 550.

FIG. 21 illustrates an embodiment of a drug delivery device 610 which has similarities in structure and/or function to the drug delivery device 510 in FIG. 20. Details of the structure and/or function that differentiate the drug delivery device 610 in FIG. 21 from the drug delivery device 510 in FIG. 20 are discussed below.

With respect to the embodiment illustrated in FIG. 21, the plunger 626 may include a guide 674 and a central rod 690. The guide 674 may have a hollow tubular shape that is open at the proximal end and closed by a transverse wall 692 at the distal end. The transverse wall 692 may be perpendicular or substantially perpendicular to the longitudinal axis A and may define a seat for the distal end of the plunger biasing member 650. The central rod 690 may have a distal end fixed to the transverse wall 692 such that the central rod 690 and the guide 674 jointly translate and jointly rotate. The central rod 690 may extend from the transverse wall 692 in the proximal direction through the interior space of the guide 674. A proximal end of the central rod 690 may be disposed adjacent to the opening in the proximal end of the guide 674, and in some embodiments, may extend outside of the opening formed in the proximal end of the guide 674 or alternatively be disposed inside of the proximal end of the guide 674.

As illustrated in FIG. 21, the brake member 670 may be fixed to the rear housing 627. The brake member 670 may have a generally annular shape and may surround the proximal end of the central rod 690. Furthermore, a threaded inner surface 670a of the brake member 670 may threadably engage a threaded outer surface 626a of the proximal end of the central rod 690. As a consequence of this threaded coupling, movement of the plunger 626, including its central rod 690, in the distal direction may cause the plunger 626 to rotate. Rotation of the plunger 626 may continue for as long as the threaded outer surface 626a of the central rod 690 remains in contact with the threaded inner surface 670a of the brake member 670. In some embodiments, rotation of the plunger 626 may cease simultaneously or nearly simultaneously with the plunger 626 striking the stopper in the drug storage container 620.

In the pre-delivery or storage state (seen in FIG. 21), the plunger 626 may be prevented from moving in the distal direction under the biasing force of the plunger biasing member 650. As an example, the drug delivery device 610 may include a lock 672 which has an initial position (FIG. 21) in which the lock 672 prevents movement of the plunger 626 in the distal direction and a second position in which the lock 672 does not prevent movement of the plunger 626 in the distal direction. As a more specific example, the lock 672 may include: a proximal end fixed to the rear housing 627; and a distal end having an initial position in which the distal end is secured to the proximal end of the guide 674 thereby preventing distal movement of the plunger 626 and a second position that is radially outward of the initial position in which the distal end does not contact the proximal end of the guide 674 thereby permitting distal movement of the plunger 626. The distal end of the lock 672 may be operably coupled to the guard member 632 such that upon retraction of the guard member 632 in the proximal direction the guard member 632 may directly or indirectly act on the distal end of the lock 672 causing it to transition from the initial position to the second position. In some embodiments, this movement of the distal end of the lock 672 may a result of a camming action between the distal end of the lock 672 and a proximal end of the guard member 632. When the lock 672 is in the second position, the plunger biasing member 650 may be allowed to expand, thereby driving the plunger 626 in the distal direction, which, in turn, causes rotation of the plunger 626 for at least a portion of the plunger stroke due to the threaded coupling between the plunger 626 and the brake member 670.

While the embodiments described above in connection with FIGS. 17A-21 utilize a threaded coupling between the plunger and the brake member for causing relative rotation between the plunger and brake member during axial translation of the plunger, other embodiments may achieve this rotation via other means. For example, the plunger and the brake member may include one or more cooperating camming surfaces which interact with each other to convert relative axial movement into a combination of relative axial movement and relative rotational movement. Furthermore, in some embodiments, resistance to distal movement of the plunger may be achieved via an air damper operably coupled to the plunger. In certain such embodiments, the plunger may not rotate when moving in the distal direction.

As will be recognized, the devices and methods according to the present disclosure may have one or more advantages relative to conventional technology, any one or more of which may be present in a particular embodiment in accordance with the features of the present disclosure included in that embodiment. Other advantages not specifically listed herein may also be recognized as well.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a housing;
a drug storage container fixed relative to the housing and including an interior surface and a stopper slidable along the interior surface;
a biasing member; and
a plunger operably coupled to the biasing member and configured to:
selectively rotate from an initial rotational position to a second rotational position under a biasing force exerted by the biasing member, and
translate linearly in a distal direction to drive the stopper through the drug storage container after rotating from the initial rotational position to the second rotational position.

2. The drug delivery device of claim 1, comprising a biasing member disposed at least partially within the plunger.

3. The drug delivery device of claim 2, the biasing member comprising a compression spring.

4. The drug delivery device of claim 3, wherein the plunger is configured to translate linearly in the distal direction while rotating from the initial rotational position to the second rotational position.

5. (canceled)

6. The drug delivery device of claim 4, comprising a plunger guide fixed relative to the housing, the plunger being disposed at least partially within the plunger guide.

7. The drug delivery device of claim 6, wherein one of the plunger and the plunger guide comprises a cam and the other one of the plunger and the plunger guide comprises a cam follower.

8. The drug delivery device of claim 7, wherein the biasing force of the biasing member urges the cam follower against the cam to urge the plunger to rotate from the initial rotational position toward the second rotational position.

9. The drug delivery device of claim 8, wherein the plunger includes the cam follower and the plunger guide includes the cam, and wherein the cam follower is formed by at least one projection extending outwardly from the plunger.

10. The drug delivery device of claim 9, wherein the plunger guide comprises an annular wall, wherein the cam is formed by a proximally facing surface of the annular wall.

11. (canceled)

12. The drug delivery device of claim 6, comprising:
a releaser member operably coupled to the plunger and configured to selectively rotate relative to the housing, wherein each of the plunger and the plunger guide is disposed at least partially within the releaser member; and
a guard moveably positioned adjacent to an opening in the housing and operably coupled to the releaser member.

13. The drug delivery device of claim 12, wherein the guard has an extended position wherein the guard extends at least partially through the opening in the housing and a retracted position wherein the guard is positioned away from the extended position toward the housing.

14. The drug delivery device of claim 13, wherein the releaser member is prevented from rotating in at least one rotational direction when the guard is in the extended position, and wherein the releaser member is allowed to rotate in the at least one rotational direction when the guard is in the retracted position.

15. The drug delivery device of claim 14, wherein moving the guard from the extended position to the retracted position allows the releaser member and the plunger to rotate jointly from the initial rotation position toward the second rotation position under the biasing force exerted by the biasing member.

16-30. (canceled)

31. A drug delivery device comprising:
a housing having an opening;
a drug storage container including a delivery member having an insertion end configured to extend at least partially through the opening;
a guard moveably positioned adjacent to the opening;
a plunger moveable in a distal direction to expel a drug from the drug storage container through the delivery member;
a plunger biasing member; and
a releaser member operably coupled to the guard and the plunger, wherein the releaser member is configured to rotate from an initial rotational position to a second rotational position under a biasing force exerted by the plunger biasing member.

32. The drug delivery device of claim 31, wherein the guard has an extended position wherein the guard extends at least partially through the opening in the housing and a retracted position wherein the guard is positioned away from the extended position toward the housing.

33. The drug delivery device of claim 32, wherein the releaser member is prevented from rotating from the initial rotational position toward the second rotation position when the guard is in the extended position, and wherein the releaser member is allowed to rotate from the initial rotational position toward the second rotational position when the guard is in the retracted position.

34. The drug delivery device of claim 33, wherein moving the guard from the extended position to the retracted position allows the releaser member and the plunger to rotate jointly from the initial rotation position to the second rotation position under the biasing force exerted by the plunger biasing member.

35. The drug delivery device of claim 34, comprising a guard extension, wherein the releaser member is disposed at least partially within the guard extension.

36. The drug delivery device of claim 35, comprising a first projection extending outwardly from the releaser member and a second projection extending inwardly from the guard extension, wherein the first and second projections engage one another to retain the releaser member in the initial rotational position.

37. The drug delivery device of claim 36, wherein the second projection slides out of engagement with the first projection to allow the releaser member to rotate away from the initial rotational position toward the second rotational position when the guard is in the retracted position.

38-53. (canceled)

54. A drug delivery device comprising:
a housing having an opening;
a drug storage container including a delivery member having an insertion end configured to extend at least partially through the opening;
a plunger;
a plunger biasing member initially retained in an energized state, wherein releasing the plunger biasing member drives the plunger in a distal direction to expel a drug from the drug storage container through the delivery member; and
an indicator having an initial position wherein the indicator retains the plunger biasing member in the energized state, and a second position wherein the indicator generates an audible signal indicating an end of drug delivery.

55. (canceled)

56. (canceled)

57. The drug delivery device of claim 54, comprising an indicator biasing member configured to bias the indicator in the proximal direction.

58. The drug delivery device of claim 57, comprising a cam and a cam follower, wherein the indicator includes the cam follower.

59. The drug delivery device of claim 58, wherein a biasing force of the indicator biasing member urges the cam follower against the cam to urge the indicator to rotate relative to the housing.

60. The drug delivery device of claim 59, wherein the indicator, when moving from the initial position to the second position, rotates relative to the housing and translates linearly in the proximal direction.

61-116. (canceled)
